(19) United States
(12) Patent Application Publication
(10) Pub. No.: US 2011/0263566 A1
(43) Pub. Date: Oct. 27, 2011

(54) 7-Hydroxy-benzoimidazole-4-yl-methanone Derivatives and PBK Inhibitors Containing the Same

(76) Inventors: Yo Matsuo, Kanagawa (JP); Yingfu Li, Clifton Park, NY (US); Joel Walker, Schenectady, NY (US); Feryan Ahmed, Latham, NY (US); Ryuji Ohsawa, Kanagawa (JP); Shoji Hisada, Kanagawa (JP)

(21) Appl. No.: 13/126,741

(22) PCT Filed: Jul. 30, 2009
(86) PCT No.: PCT/US09/52228

§ 371 Date: Jul. 12, 2011

(30) Foreign Application Priority Data
Oct. 30, 2008 (US) 61109801

(51) Int. Cl. A61K 31/4184; A61K 31/454; C07D 403/12; A61P 35/00; C07D 409/14; A61K 31/496; C07D 235/06; C07D 401/12; C07D 409/04

(52) U.S. Cl. 514/210.21; 546/199; 514/322; 548/306.1; 514/394; 548/304.7; 544/370; 514/254.06; 548/304.4; 544/364; 514/253.09

(57) ABSTRACT

7-Hydroxy-benzoimidazole-4-yl-methanone Derivatives, which are useful for PBK inhibitors, are provided.

### PRIORITY

The present application claims the benefit of U.S. Provisional Application No. 61/109,801, filed on Oct. 30, 2008, the entire contents of which are incorporated by reference herein.

### TECHNICAL FIELD

The present invention relates to a compound for inhibiting PBK activity, a method for the preparation thereof, and a pharmaceutical composition containing the compound as an active ingredient.

### BACKGROUND ART

Previous studies revealed that PDZ binding kinase (PBK) is a serine/threonine kinase related to the dual specific mitogen-activated protein kinase kinase (MAPKK) family (Abe Y, et al., J Biol. Chem. 275: 21525-21531, 2000, Gaudet S, et al., Proc Natl Acad. Sci. 97: 5167-5172, 2000 and Matsumoto S, et al., Biochem Biophys Res Commun. 325: 997-1004, 2004). PBK was also indicated to be involved in mitosis as shown by its significant role in highly proliferating spermatocytes (Gaudet S, et al., Proc Natl Acad. Sci. 97: 5167-5172, 2000 and Fujibuchi T, et al., Dev Growth Differ. 47:637-44, 2005). In fact, abundant expression of PBK was observed in testis, while almost no PBK expression was detected in other normal organs (Park J H, et al., Cancer Res. 66: 9186-95, 2006). PBK regulates cell cycle progression. In accordance with this, its significant overexpression was detected in clinical breast cancer samples (Park J H, et al., Cancer Res. 66: 9186-95, 2006), Burkitt's lymphoma (Simons-Evelyn M, et al., Blood Cells Mol. Dis. 27: 825-829, 2001) and a variety of hematologic malignancies (Nandi A, et al., Blood Cells Mol. Dis. 32: 240-5, 2004).

Immunohistochemical analysis of testis revealed the expression of PBK protein around the outer region of seminiferous tubules where repeated mitosis of sperm germ cells followed by meiosis occurs (Fujibuchi T, et al., Dev Growth Differ. 47: 637-44, 2005). Especially, at prophase and metaphase, the subcellular localization of PBK was detected around the condensed chromosome in breast cancer cells (Park J H, et al., Cancer Res. 66: 9186-95, 2006). Moreover the knockdown of PBK expression with gene specific siRNAs caused dysfunction of cytokinesis and subsequently led to apoptosis of the cancer cells (Park J H, et al., Cancer Res. 66: 9186-95, 2006). These indicated the critical function of PBK at mitosis, in testicular and cancer cells.

Taken together, PBK-specific inhibitors can be used as a drug applicable for a broad spectrum of cancers. PBK is an excellent target for cancer therapy for the following reasons: i) almost no expression in normal organs (except for testis); ii) frequent overexpression in clinical cancer samples; iii) a serine/threonine kinase related to the essential function for cell mitosis.

The present inventors have endeavored to develop an effective inhibitor of PBK and have found that a 7-hydroxy-benzoimidazole-4-yl-methanone derivative can selectively inhibit the activity of PBK.

### SUMMARY OF INVENTION

Accordingly, it is an object of the present invention to provide a PBK inhibitor having high inhibitory activity against PBK.

It is another object of the present invention to provide a method for preparing such inhibitor.

It is a further object of the present invention to provide a pharmaceutical composition including the compound, a pharmaceutically acceptable salt, hydrate, solvate, or isomer thereof.

In accordance with one aspect of the present invention, there is provided a compound of formula (I), and a pharmaceutically acceptable salt, hydrate, solvate, or isomer thereof:

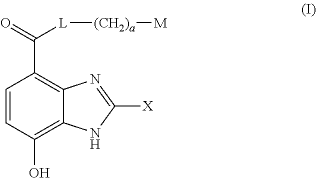

wherein
X is phenyl, thiophen-2-yl, furan-2-yl, cyclopropyl, cyclopentyl, phenylC₁-C₆ alkyl, thiophen-2-ylC₁-C₆ alkyl, furan-2-ylC₁-C₆ alkyl, cyclopropylC₁-C₆ alkyl, cyclopentylC₁-C₆ alkyl, or bicycle[2.2.1]heptan-2-yl;
   the phenyl, thiophen-2-yl, furan-2-yl, cyclopropyl, cyclopentyl, phenylC₁-C₆alkyl, thiophen-2-ylC₁-C₆ alkyl, furan-2-ylC₁-C₆ alkyl, cyclopropylC₁-C₆ alkyl, or cyclopentylC₁-C₆ alkyl are optionally substituted by 1-3 substituent(s) each independently selected from group A;
   L is —NH— or a single bond;
   M is selected from C₃-C₁₀ cycloalkyl or 3-10 membered saturated heterocyclic group;
   the C₃-C₁₀ cycloalkyl, and 3-8 membered saturated heterocyclic group are optionally substituted by 1-3 substituent(s) each independently selected from group A;
wherein group A consists of hydroxyl, oxo, nitro, cyano, amino, C₁-C₆ alkylamino, C₃-C₁₀ cycloalkylamino, amide, halogen, sulfamoyl, trifluolomethyl, p-toluenesulfonylamino, C₁-C₆ alkyl, C₃-C₁₀ cycloalkyl, C₁-C₆ alkoxy, C₁-C₆ alkoxycarbonyl, C₁-C₆ alkylcarbonylamino, C₁-C₆ alkylsulfonyl, C₁-C₆ alkylsulfonylamino, C₁-C₆ alkenyl, C₁-C₆ alkynyl, phosphoryl, carbonyl, carboxyl, and 3-8 membered saturated heterocyclic group; and
   a is an integer from 0-5.

### DESCRIPTION OF EMBODIMENTS

### Definition

In this invention, “alkyl” refers to a straight chain or a branched chain hydrocarbon group which does not contain any hetero atoms or unsaturated carbon-carbon bonds. “C₁-C₆ alkyl” refers to an alkyl group which has 1-6 carbon atom(s). “C₁-C₄ alkyl” refers to an alkyl group which has 1-4 carbon atom(s).

Examples of “C₁-C₆ alkyl” include, but are not limited to, methyl, ethyl, 1-propyl, 2-propyl, 2-methyl-1-propyl, 2-methyl-2-propyl (tert-butyl(1,1-dimethyl-ethyl), 1-butyl, 2-butyl, 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,3-dimethyl-1-butyl, 3,3-dimethyl-1-butyl, 2,2-dimethyl-1-butyl, 2-ethyl-1-butyl, 3,3-dimethyl-2-butyl, and 2,3-dimethyl-2-butyl.

In this invention, “phenylC₁-C₆ alkyl, thiophen-2-ylC₁-C₆ alkyl, furan-2-ylC₁-C₆ alkyl, cyclopropylC₁-C₆ alkyl, or cyclopentylC₁-C₆ alkyl” refers to the C₁-C₆ alkyl bound to a phenyl, thiophen-2-yl, furan-2-yl, cyclopropyl or cyclopentyl group. In one embodiment, phenylC₁-C₆ alkyl, thiophen-2-ylC₁-C₆ alkyl, furan-2-ylC₁-C₆ alkyl, cyclopropylC₁-C₆ alkyl, or cyclopentylC₁-C₆ alkyl is optionally substituted by 1-3 substituent(s) each independently selected from the group A mentioned above. Such substitution may occur at either the phenyl, thiophen-2-yl, furan-2-yl, cyclopropyl, or cyclopentyl moiety or the C₁-C₆ alkyl moiety of said group, or may occur at both moieties of said group.

Examples of “phenylC₁-C₆ alkyl, thiophen-2-ylC₁-C₆ alkyl, furan-2-ylC₁-C₆ alkyl, cyclopropylC₁-C₆ alkyl, or cyclopentylC₁-C₆ alkyl” include, but are not limited to, phenylmethyl, phenylethyl, phenyl-1-propyl, phenyl-2-propyl, phenyl-n-butyl, phenyl-s-butyl, phenyl-t-butyl, phenyl-2-ethylbutyl, thiophen-2-ylmethyl, thiophen-2-ylethyl, thiophen-2-yl-1-propyl, thiophen-2-yl-2-propyl, thiophen-2-yl-n-butyl, thiophen-2-yl-s-butyl, thiophen-2-yl-t-butyl, thiophen-2-yl-2-ethylbutyl, furan-2-ylmethyl, furan-2-ylethyl, furan-2-yl-1-propyl, furan-2-yl-2-propyl, furan-2-yl-n-butyl, furan-2-yl-s-butyl, furan-2-yl-t-butyl, furan-2-yl-2-ethylbutyl, cyclopropylmethyl, cyclopropylethyl, cyclopropyl-1-propyl, cyclopropyl-2-propyl, cyclopropyl-n-butyl, cyclopropyl-s-butyl, cyclopropyl-t-butyl, cyclopropyl-2-ethylbutyl, cyclopentylmethyl, cyclopentylethyl, cyclopentyl-1-propyl, cyclopentyl-2-propyl, cyclopentyl-n-butyl, cyclopentyl-s-butyl, cyclopentyl-t-butyl and cyclopentyl-2-ethylbutyl.

In this invention, “alkenyl” refers to a straight chain or a branched chain hydrocarbon group which contains one or more than one unsaturated carbon-carbon bond(s) and does not contain any hetero atoms. “C₂-C₆ alkenyl” refers to an alkenyl group which has 2-6 carbon atoms.

Examples of “C₂-C₆ alkenyl” include, but are not limited to, vinyl(ethenyl), 1-propenyl, 2-propenyl, 3-propenyl, 2-methyl-prop-1-en-1-yl (2-methyl-1-propenyl), 2-methyl-prop-1-en-3-yl (2-methyl-2-propenyl), but-1-en-1-yl, but-1-en-2-yl, but-1-en-3-yl, but-2-en-1-yl, but-2-en-2-yl, pent-1-en-1-yl, pent-1-en-2-yl, pent-1-en-3-yl, pent-1-en-4-yl, pent-1-en-5-yl, pent-2-en-1-yl, pent-2-en-2-yl, pent-2-en-3-yl (1-ethyl-1-propenyl), pent-2-en-4-yl, pent-2-en-5-yl, 2-methyl-but-1-en-1-yl, 2-methyl-but-1-en-2-yl, 2-methyl-but-1-en-3-yl, 2-methyl-but-1-en-4-yl, 2-methyl-but-2-en-1-yl, 2-methyl-but-2-en-3-yl, 2-methyl-but-2-en-4-yl, 3-methyl-but-1-en-1-yl, 3-methyl-but-1-en-2-yl, 3-methyl-but-1-en-3-yl, 3-methyl-but-1-en-4-yl, 2,2-dimethyl-prop-1-en-1-yl, 2,2-dimethyl-prop-1-en-2-yl, hex-1-en-1-yl, hex-1-en-2-yl, hex-1-en-3-yl, hex-1-en-4-yl, hex-1-en-5-yl, hex-1-en-6-yl, hex-2-en-1-yl, hex-2-en-2-yl, hex-2-en-3-yl, hex-2-en-4-yl, hex-2-en-5-yl, hex-2-en-6-yl, hex-3-en-1-yl, hex-3-en-2-yl, hex-3-en-3-yl, 2-methyl-pent-1-en-1-yl, 2-methyl-pent-1-en-3-yl, 2-methyl-pent-1-en-4-yl, 2-methyl-pent-1-en-5-yl, 2-methyl-pent-2-en-1-yl, 2-methyl-pent-2-en-3-yl, 2-methyl-pent-2-en-4-yl, 2-methyl-pent-2-en-5-yl, 3-methyl-pent-1-en-1-yl, 3-methyl-pent-1-en-2-yl, 3-methyl-pent-1-en-3-yl, 3-methyl-pent-1-en-4-yl, 3-methyl-pent-1-en-5-yl, 3-methyl-pent-2-en-1-yl, 3-methyl-pent-2-en-2-yl, 3-methyl-pent-2-en-4-yl, 3-methyl-pent-2-en-5-yl, 4-methyl-pent-1-en-1-yl, 4-methyl-pent-1-en-2-yl, 4-methyl-pent-1-en-3-yl, 4-methyl-pent-1-en-4-yl, 4-methyl-pent-1-en-5-yl, 4-methyl-pent-2-en-1-yl, 4-methyl-pent-2-en-2-yl, 4-methyl-pent-2-en-3-yl, 4-methyl-pent-2-en-4-yl, 4-methyl-pent-2-en-5-yl, 2,3-dimethyl-but-1-en-1-yl, 2,3-dimethyl-but-1-en-3-yl, 2,3-dimethyl-but-1-en-4-yl, 2,3-dimethyl-but-2-en-1-yl, 3,3-dimethyl-but-1-en-1-yl, 3,3-dimethyl-but-1-en-2-yl, 3,3-dimethyl-but-1-en-4-yl, 2-ethyl-but-1-en-1-yl, 2-ethyl-but-1-en-3-yl, 2-ethyl-but-1-en-4-yl, 3-ethyl-but-1-en-1-yl, 3-ethyl-but-1-en-2-yl, 3-ethyl-but-1-en-3-yl, 3-ethyl-but-1-en-4-yl, 2-ethyl-but-2-en-1-yl, 2-ethyl-but-2-en-3-yl and 2-ethyl-but-2-en-4-yl.

In this invention, “alkynyl” refers to a straight chain or a branched chain hydrocarbon group which contains at least one triple carbon-carbon bond and does not contain any hetero atoms. “C₂-C₆ alkynyl” refers to an alkynyl group which has 2-6 carbon atoms.

Examples of “C₂-C₆ alkynyl” include, but are not limited to, ethinyl, 1-propinyl, 2-propinyl, 3-propinyl, 2-methyl-prop-1-in-1-yl, 2-methyl-prop-1-in-3-yl, but-1-in-1-yl, but-1-in-2-yl, but-1-in-3-yl, but-2-in-1-yl, but-2-in-2-yl, pent-1-in-1-yl, pent-1-in-2-yl, pent-1-in-3-yl, pent-1-in-4-yl, pent-1-in-5-yl, pent-2-in-1-yl, pent-2-in-2-yl, pent-2-in-3-yl, pent-2-in-4-yl, pent-2-in-5-yl, 2-methyl-but-1-in-1-yl, 2-methyl-but-1-in-2-yl, 2-methyl-but-1-in-3-yl, 2-methyl-but-1-in-4-yl, 2-methyl-but-2-in-1-yl, 2-methyl-but-2-in-3-yl, 2-methyl-but-2-in-4-yl, 3-methyl-but-1-in-1-yl, 3-methyl-but-1-in-2-yl, 3-methyl-but-1-in-3-yl, 3-methyl-but-1-in-4-yl, 2,2-dimethyl-prop-1-in-1-yl, 2,2-dimethyl-prop-1-in-2-yl, hex-1-in-1-yl, hex-1-in-2-yl, hex-1-in-3-yl, hex-1-in-4-yl, hex-1-in-5-yl, hex-1-in-6-yl, hex-2-in-1-yl, hex-2-in-2-yl, hex-2-in-3-yl, hex-2-in-4-yl, hex-2-in-5-yl, hex-2-in-6-yl, hex-3-in-1-yl, hex-3-in-2-yl, hex-3-in-3-yl, 2-methyl-pent-1-in-1-yl, 2-methyl-pent-1-in-3-yl, 2-methyl-pent-1-in-4-yl, 2-methyl-pent-1-in-5-yl, 2-methyl-pent-2-in-1-yl, 2-methyl-pent-2-in-3-yl, 2-methyl-pent-2-in-4-yl, 2-methyl-pent-2-in-5-yl, 3-methyl-pent-1-in-1-yl, 3-methyl-pent-1-in-2-yl, 3-methyl-pent-1-in-3-yl, 3-methyl-pent-1-in-4-yl, 3-methyl-pent-1-in-5-yl, 3-methyl-pent-2-in-1-yl, 3-methyl-pent-2-in-2-yl, 3-methyl-pent-2-in-4-yl, 3-methyl-pent-2-in-5-yl, 4-methyl-pent-1-in-1-yl, 4-methyl-pent-1-in-2-yl, 4-methyl-pent-1-in-3-yl, 4-methyl-pent-1-in-4-yl, 4-methyl-pent-1-in-5-yl, 4-methyl-pent-2-in-1-yl, 4-methyl-pent-2-in-2-yl, 4-methyl-pent-2-in-3-yl, 4-methyl-pent-2-in-4-yl, 4-methyl-pent-2-in-5-yl, 2,3-dimethyl-but-1-in-1-yl, 2,3-dimethyl-but-1-in-3-yl, 2,3-dimethyl-but-1-in-4-yl, 2,3-dimethyl-but-2-in-1-yl, 3,3-dimethyl-but-1-in-1-yl, 3,3-dimethyl-but-1-in-2-yl, 3,3-dimethyl-but-1-in-4-yl, 2-ethyl-but-1-in-1-yl, 2-ethyl-but-1-in-3-yl, 2-ethyl-but-1-in-4-yl, 3-ethyl-but-1-in-1-yl, 3-ethyl-but-1-in-2-yl, 3-ethyl-but-1-in-3-yl, 3-ethyl-but-1-in-4-yl, 2-ethyl-but-2-in-1-yl, 2-ethyl-but-2-in-3-yl and 2-ethyl-but-2-in-4-yl.

In the present invention, “alkoxy” refers to a group represented by —OR, wherein R is alkyl.

“C₁-C₆ alkoxy” refers to an alkoxy group which has 1-6 carbon atom(s). “C₁-C₄ alkoxy” refers to an alkoxy group which has 1-4 carbon atom(s).

Examples of “C₁-C₆ alkoxy” include, but are not limited to, methoxy, ethoxy, 1-propyloxy, 2-propyloxy, 2-methyl-1-propyloxy, 2-methyl-2-propyloxy, and 1-butyloxy, and 2-butyloxy.

In this invention, “C₁-C₆ alkylcarbonyl” refers to R—C═O— wherein R is C₁-C₆alkyl. “C₁-C₄ alkylcarbonyl” refers to R—C═O— wherein R is C₁-C₄alkyl.

Examples of “C₁-C₆ alkylcarbonyl” include, but are not limited to, methylcarbonyl (acetyl), ethylcarbonyl, propylcarbonyl, iso-propylcarbonyl, n-butylcarbonyl, s-butylcarbonyl, t-butylcarbonyl, and 2-ethylbutylcarbonyl.

In this invention, “C₁-C₆ alkoxycarbonyl” refers to a carbonyl group bound to the C₁-C₆ alkoxy. “C₁-C₄ alkoxycarbonyl” refers to a carbonyl group bound to the C₁-C₄ alkoxy.

Examples of “C₁-C₆ alkoxycarbonyl” include, but are not limited to, methoxycarbonyl, ethoxycarbonyl, propoxycarbonyl, and t-butoxycarbonyl.

In the present invention, “cycloalkyl” refers to a saturated carbon ring system. “C₃-C₁₀ cycloalkyl” refers to 3-10 membered cycloalkyl.

Examples of “C₃-C₁₀ cycloalkyl” include, but are not limited to, cyclopropyl, cyclobutyl, cyclopentyl, cyclohexyl, cycloheptanyl, cyclooctanyl, and adamantyl. For example, 3-8 membered cycloalkyl is also included in “C₃-C₁₀ cycloalkyl”.

In this invention, “amino” refers to a group represented by —NH₂ whose hydrogens may each be optionally substituted by a substituent.

In the present invention, “C₁-C₆ alkylamino” refers to an amino group bound to the C₁-C₆ alkyl.

Examples of “C₁-C₆ alkylamino” include, but are not limited to, methylamino, ethylamino, propylamino, isopropylamino, n-butylamino, s-butylamino, t-butylamino, and 2-ethylbutylamino.

In the present invention, “C₁-C₆ alkylcarbonylamino” refers to R—C═O—NH— wherein R is C₁-C₆ alkyl. “C₁-C₄ alkylcarbonylamino” refers to R—C═O—NH— wherein R is C₁-C₄ alkyl.

Examples of “C₁-C₆ alkylcarbonylamino” include, but are not limited to, methylcarbonylamino (acetyl amino), ethylcarbonylamino, 1-propylcarbonylamino, 2-propylcarbonylamino, n-butylcarbonylamino, s-butylcarbonylamino, t-butylcarbonylamino, and 2-ethylbutylcarbonylamino.

In the present invention, “C₃-C₁₀ cycloalkylamino” refers to R—NH— wherein R is C₃-C₁₀cycloalkyl.

Examples of “C₃-C₁₀ cycloalkyl amino” include, but are not limited to, cyclopropylamino, cyclobutylamino, cyclopentylamino, cyclohexylamino, cycloheptanylamino, and cyclooctanyl amino.

In this invention, “sulfonyl” is a group represented by —SO₂—.

In this invention, “C₁-C₆ alkylsulfonyl” refers to R—SO₂— wherein R is the C₁-C₆ alkyl. “C₁-C₄ alkylsulfonyl” refers to R—SO₂— wherein R is C₁-C₄ alkyl.

Examples of “C₁-C₆ alkylsulfonyl” include, but are not limited to, methylsulfonyl, ethylsulfonyl, propylsulfonyl, isopropylsulfonyl, n-butylsulfonyl, s-butylsulfonyl, t-butylsulfonyl, and 2-ethylbutylsulfonyl.

In the present invention, “C₁-C₆alkylsulfonylamino” refers to R—SO₂—NH— wherein R is “C₁-C₆ alkyl”. “C₁-C₄ alkylsulfonylamino” refers to R—SO₂—NH— wherein R is R—SO₂—NH— wherein R is “C₁-C₄ alkyl”.

Examples of “C₁-C₆ alkylsulfonylamino” include, but are not limited to, methylsulfonylamino, ethylsulfonylamino, propylsulfonylamino, isopropylsulfonylamino, n-butylsulfonylamino, s-butylsulfonylamino, t-butylsulfonylamino, and 2-ethylbutylsulfonylamino.

In the present invention, “a saturated heterocyclic group” refers to a saturated heterocyclic group having one or more than one hetero atom(s) in the ring system. “3-8 membered saturated heterocyclic group” refers to a saturated heterocyclic group whose ring consists of 3-8 atoms.

Examples of “3-8 membered saturated heterocyclic group” include, but are not limited to, aziridinyl, azetidinyl, pyrrolidinyl, imidazolidinyl, piperazinyl, piperidinyl, azepanyl, and morpholinyl.

A salt is defined as the product formed from the neutralisation reaction of acids and bases. Salts are ionic compounds composed of cations (positively charged ions) and anions (negative ions) so that the product is electrically neutral. These component ions can be inorganic as well as organic.

Hydrate is a term used in inorganic chemistry and organic chemistry to indicate that a substance contains water. Solvate refers to a molecule in a solution complexed by solvent molecules.

Isomers are compounds with the same molecular formula but different structural formulae. More specifically, isomer includes geometric isomer, optical isomer, stereoisomer, tautomer of the compound, and mixtures thereof.

The present invention provides a compound represented by formula (I):

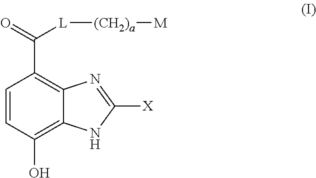

wherein
X is phenyl, thiophen-2-yl, furan-2-yl, cyclopropyl, cyclopentyl, phenylC₁-C₆alkyl, thiophen-2-ylC₁-C₆ alkyl, furan-2-ylC₁-C₆ alkyl, cyclopropylC₁-C₆ alkyl, cyclopentylC₁-C₆ alkyl, or bicycle[2.2.1]heptan-2-yl;
   the phenyl, thiophen-2-yl, furan-2-yl, cyclopropyl, cyclopentyl, phenylC₁-C₆alkyl, thiophen-2-ylC₁-C₆alkyl, furan-2-ylC₁-C₆alkyl, cyclopropylC₁-C₆ alkyl, or cyclopentylC₁-C₆ alkyl are optionally substituted by 1-3 substituent(s) each independently selected from group A;
L is —NH— or single bond;
M is selected C₃-C₁₀ cycloalkyl or 3-8 membered saturated heterocyclic group;
   the C₃-C₁₀ cycloalkyl, and 3-8 membered saturated heterocyclic group are optionally substituted by 1-3 substituent(s) each independently selected from group A;
wherein group A consists of hydroxyl, oxo, nitro, cyano, amino, C₁-C₆ alkylamino, C₃-C₁₀ cycloalkylamino, amide, halogen, sulfamoyl, trifluolomethyl, p-toluenesulfonylamino, C₁-C₆ alkyl, C₃-C₁₀ cycloalkyl, C₁-C₆alkoxy, C₁-C₆alkoxycarbonyl, C₁-C₆ alkylcarbonylamino, C₁-C₆ alkylsulfonyl, C₁-C₆ alkylsulfonylamino, C₁-C₆alkenyl, C₁-C₆alkynyl, phosphoryl, carbonyl, carboxyl, and 3-8 membered saturated heterocyclic group; and
   a is an integer from 0-5.

Preferred compounds include those selected from the group consisting of: Example Nos. 5, 6, 7, 8, 9, 10, 11, 12, 13, 14, 15, 16, 17, 18, 19, 20, 21, 35, 36, 37, 38, 39, 40, 41, 42, 43, 44, 45, 46, 47, 48, 49, 50, 51, 52, 53, 54, 55, 56, 57, 58, 59, and 60 listed in Table 1 below; and the pharmaceutically acceptable salts, prodrugs, hydrates and solvates of the forgoing compounds.

TABLE 1

| Example No. | Structure | Compound |
|---|---|---|
| 5 | 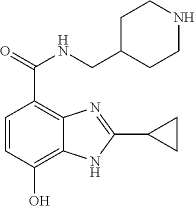 | 2-Cyclopropyl-4-hydroxy-N-(piperidin-4-ylmethyl)-1H- benzo[d]imidazole-7-carboxamide |
| 6 | 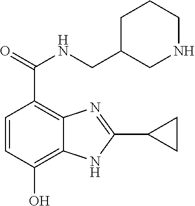 | 2-Cyclopropyl-4-hydroxy-N-(piperidin-3-yl-methyl)-1H- benzo[d]imidazole-7-carboxamide |
| 7 | 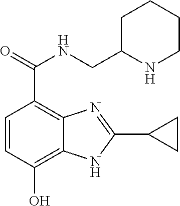 | 2-Cyclopropyl-4-hydroxy-N-(piperidin-2-ylmethyl)-1H- benzo[d]imidazole-7-carboxamide |
| 8 | 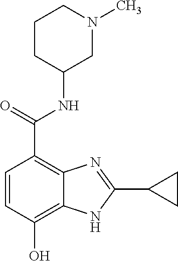 | 2-cyclopropyl-4-hydroxy-N-(1-methylpiperidin-3-yl)-1H- benzo[d]imidazole-7-carboxamide |
| 9 | 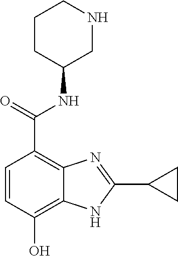 | (S)-2-cyclopropyl-4-hydroxy-N-(piperidin-3-yl)-1H- benzo[d]imidazole-7-carboxamide |
| 10 | 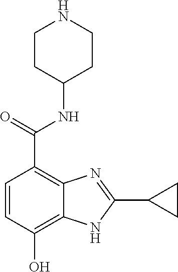 | 2-cyclopropyl-4-hydroxy-N-(piperidin-4-yl)-1H- benzo[d]imidazole-7-carboxamide |
| 11 | 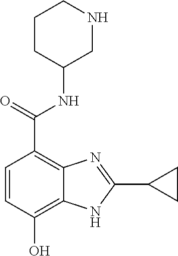 | 2-cyclopropyl-4-hydroxy-N-(piperidin-3-yl)-1H- benzo[d]imidazole-7-carboxamide |
| 12 | 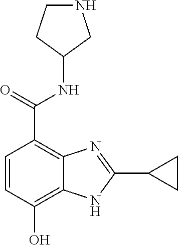 | 2-cyclopropyl-4-hydroxy-N-(pyrrolidin-3-yl)-1H- benzo[d]imidazole-7-carboxamide |
| 13 | 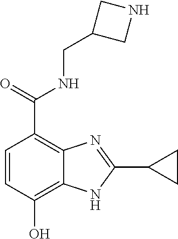 | N-(azetidin-3-ylmethyl)-2-cyclopropyl-4-hydroxy-1H- benzo[d]imidazole-7-carboxamide |
| 14 | 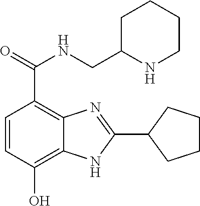 | 2-cyclopentyl-4-hydroxy-N-(piperidin-2-ylmethyl)-1H- benzo[d]imidazole-7-carboxamide |
| 15 | 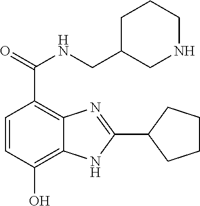 | 2-Cyclopentyl-4-hydroxy-N-(piperidin-3-ylmethyl)-1H- benzo[d]imidazole-7-carboxamide |
| 16 | 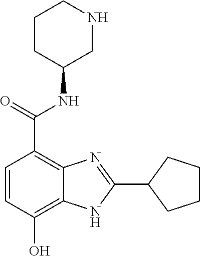 | (S)-2-Cyclopentyl-4-hydroxy-N-(piperidin-3-yl)-1H- benzo[d]imidazole-7-carboxamide |
| 17 | 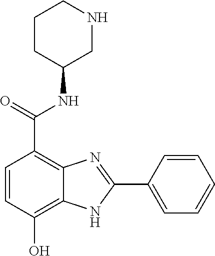 | (S)-4-Hydroxy-2-phenyl-N-(piperidin-3-yl)-1H- benzo[d]imidazole-7-carboxamide |
| 18 | 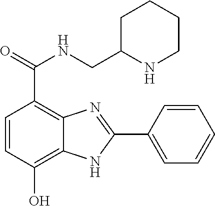 | 4-Hydroxy-2-phenyl-N-(piperidin-2-ylmethyl)-1H- benzo[d]imidazole-7-carboxamide |
| 19 | 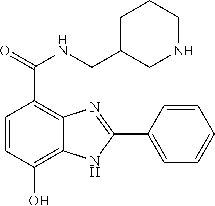 | 4-hydroxy-2-phenyl-N-(piperidin-3-ylmethyl)-1H-benzo [d]imidazole-7-carboxamide |
| 20 | 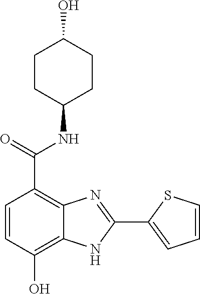 | 7-Hydroxy-N-(4-hydroxycyclohexyl)-2-(thiophen-2-yl)- 1H-benzo[d]imidazole-4-carboxamide |
| 21 | 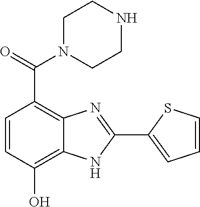 | (7-hydroxy-2-[thiophen-2-yl]-1H-benzo[d]imidazol-4-yl) (piperazin-1-yl)methanone |
| 35 | 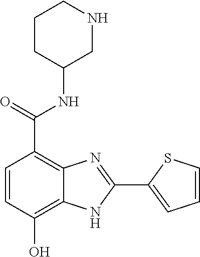 | 7-Hydroxy-N-(piperidin-3-yl)-2-(thiophen-2-yl)-1H- benzo[d]imidazole-4-carboxamide |
| 36 | 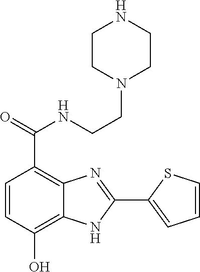 | 7-Hydroxy-N-[2-(piperazin-1-yl)ethyl]-2-(thiophen-2-yl)- 1H-benzo[d]imidazole-4-carboxamide |
| 37 | 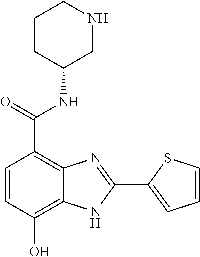 | (R)-7-Hydroxy-N-(piperidin-3-yl)-2-(thiophen-2-yl)-1H- benzo[d]imidazole-4-carboxamide |
| 38 | 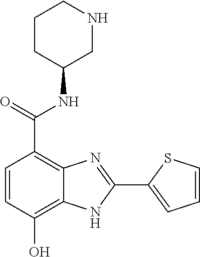 | (S)-7-Hydroxy-N-(piperidin-3-yl)-2-(thiophen-2-yl)-1H- benzo[d]imidazole-4-carboxamide |
| 39 | 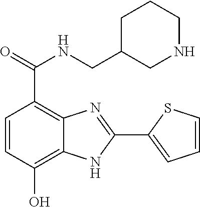 | 7-Hydroxy-N-(piperidin-3-ylmethyl)-2-(thiophen-2-yl)- 1H-benzo[d]imidazole-4-carboxamide |
| 40 | 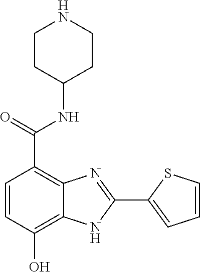 | 7-Hydroxy-N-(piperidin-4-yl)-2-(thiophen-2-yl)-1H- benzo[d]imidazole-4-carboxamide |
| 41 | 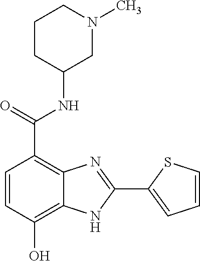 | 7-Hydroxy-N-(1-methylpiperidin-3-yl)-2-(thiophen-2-yl)- 1H-benzo[d]imidazole-4-carboxamide |
| 42 | 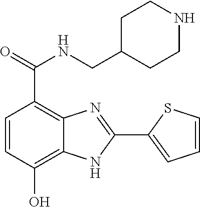 | 7-Hydroxy-N-(piperidin-4-ylmethyl)-2-(thiophen-2-yl)- 1H-benzo[d]imidazole-4-carboxamide |
| 43 | 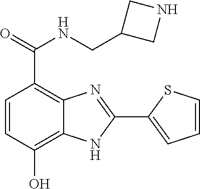 | N-(Azetidin-3-ylmethyl)-7-hydroxy-2-(thiophen-2-yl)-1H- benzo[d]imidazole-4-carboxamide |
| 44 | 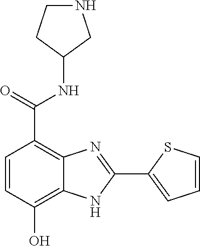 | 7-Hydroxy-N-(pyrrolidin-3-yl)-2-(thiophen-2-yl)-1H- benzo[d]imidazole-4-carboxamide |
| 45 | 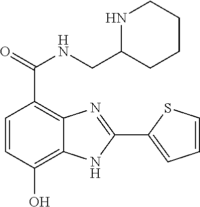 | 7-Hydroxy-N-(piperidin-2-ylmethyl)-2-(thiophen-2-yl) 1H-benzo[d]imidazole-4-carboxamide |
| 46 | 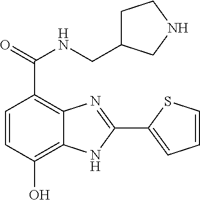 | 7-Hydroxy-N-(pyrrolidin-3-ylmethyl)-2-(thiophen-2-yl)-1H benzo[d]imidazole-4-carboxamide |
| 47 | 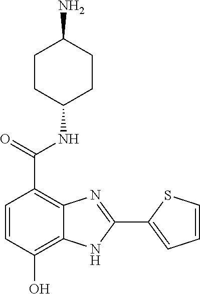 | N-(4-Aminocyclohexyl)-7-hydroxy-2-(thiophen-2-yl)-1H- benzo[d]imidazole-4-carboxamide |
| 48 | 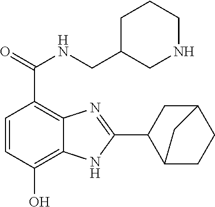 | 2-(Bicyclo[2.2.1]heptan-2-yl)-7-hydroxy-N-(piperidin-3- ylmethyl)-1H-benzo[d]imidazole-4-carboxamide |
| 49 | 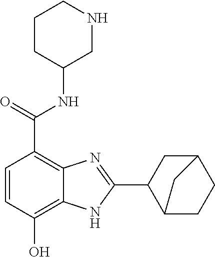 | 2-(Bicyclo[2.2.1]heptan-2-yl)-7-hydroxy-N-(piperidin-3- yl)-1H-benzo[d]imidazole-4-carboxamide |
| 50 | 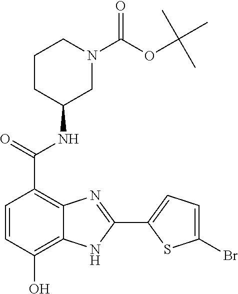 | (S)-tert-Butyl3-(2-(5-bromothiophen-2-yl)-7-hydroxy-1H- benzo[d]imidazole-4-carboxamido)piperidine-1- carboxylate |
| 51 | 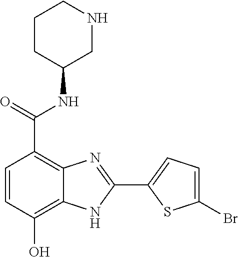 | (S)-2-(5-bromothiophen-2-yl)-7-hydroxy-N-(piperidin-3- yl)-1H-benzo[d]imidazole-4-carboxamide |
| 52 | 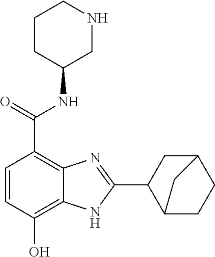 | 2-(Bicyclo[2.2.1]heptan-2-yl)-7-hydroxy-N-((S)-piperidin- 3-yl)-1H-benzo[d]imidazole-4-carboxamide |
| 53 | 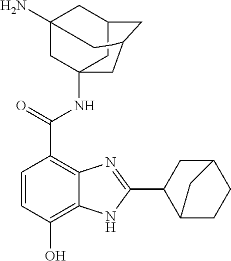 | 2-(Bicyclo[2.2.1]heptan-2-yl)-7-hydroxy-N-(adamantane- 3-ylamino)-1H-benzo[d]imidazole-4-carboxamide |
| 54 | 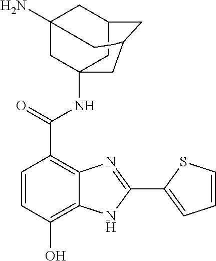 | 2-(Thiophene-2-yl)-7-hydroxy-N-(adamantate-3-ylamino)- 1H-benzo[d]imidazole-4-carboxamide |
| 55 | 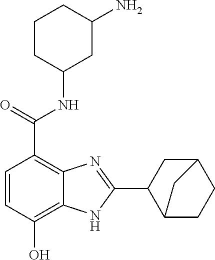 | N-(3-Aminocyclohexyl)-2-(bicyclo[2.2.1]heptan-2-yl)-7- hydroxy-1H-benzo[d]imidazole-4-carboxamide |
| 56 | 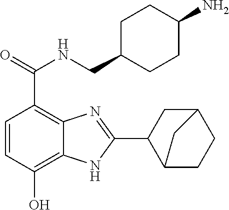 | N-{[(cis)-4-Aminocyclohexyl]methyl}-2-(bicyclo[2.2.1] heptan-2-yl)-7-hydroxy-1H-benzo[d]imidazole-4- carboxamide |
| 57 | 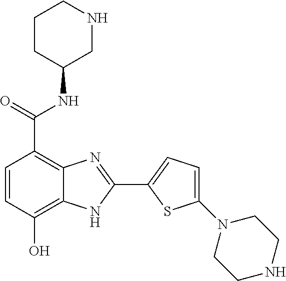 | (S)-7-hydroxy-2-(5-(piperazin-1-yl)thiophen-2-yl)-N- (piperidin-3-yl)-1H-benzo[d]imidazole-4-carboxamide |
| 58 | 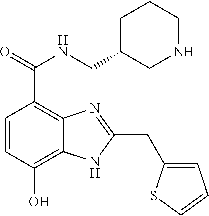 | (R)-7-hydroxy-N-(piperidin-3-ylmethyl)-2-(thiophen-2-yl methyl)-1H-benzo[d]imidazole-4-carboxamide |
| 59 | 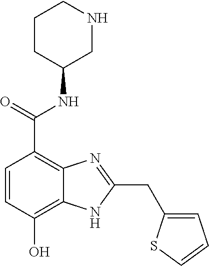 | (S)-7-hydroxy-N-(piperidin-3-yl)-2-(thiophen-2-ylmethyl)- 1H-benzo[d]imidazole-4-carboxamide |
| 60 | 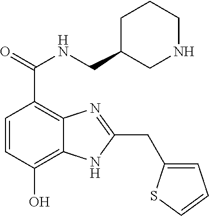 | (S)-7-hydroxy-N-(piperidin-3-ylmethyl)-2-(thiophen-2-yl methyl)-1H-benzo[d]imidazole-4-carboxamide |

The compound of formula (I) of the present invention may be in the form of a pharmaceutically acceptable salt derived from an inorganic or organic acid, and representative examples of the pharmaceutically acceptable salt derived from an inorganic or organic acid include salts obtained by adding an inorganic acid such as hydrochloric acid, hydrobromic acid, phosphoric acid or sulfonic acid, or organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid or malic acid, methanesulfonic acid, or para toluenesulfonic acid, which do not limit its scope, to the compound of formula (I). Such acids may be prepared by the conventional processes, and other acids, which themselves are not pharmaceutically acceptable, including oxalic acid may be employed in the preparation of the salts.

Alternatively, the compound of formula (I) of the present invention may also be in the form of a pharmaceutically acceptable salt derived from an inorganic or organic base include salts obtained by adding an inorganic or organic base. For example, alkalis including sodium hydroxide or potassium hydroxide, or alkaline earth metal hydroxides including calcium hydroxide, magnesium hydroxide, aluminum hydroxide or ammonium hydroxide may be used for the preparation of inorganic salt of the compound. Further, organic bases including triethylamine or diisopropylethylamine may also be used for the preparation of organic salt of the compound.

The preferred inventive compound of formula (I) may be prepared as in Scheme (I).

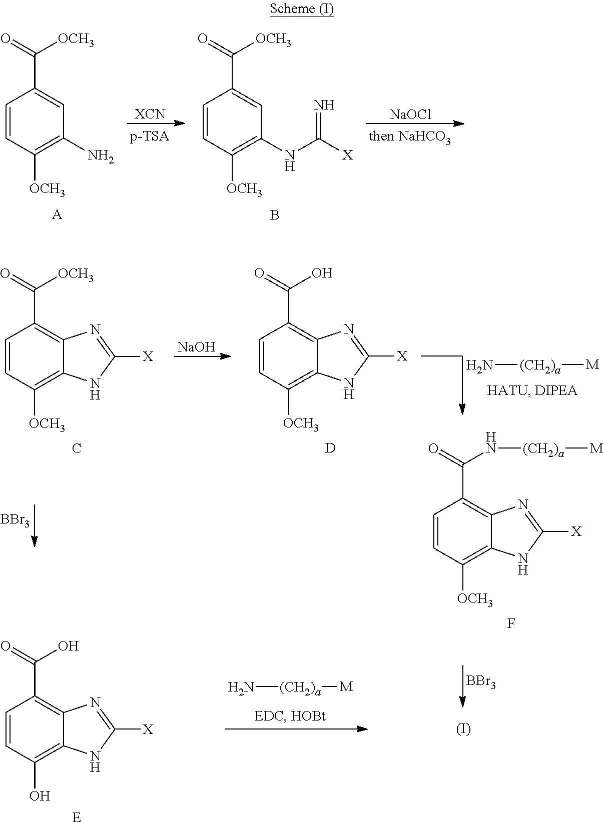

Wherein, p-TSA is p-toluenesulfonic acid, HATU is 2-(1H-7-Azabenzotriazol-1-yl)-1,1,3,3-tetramethyl uronium hexafluorophosphate Methanaminium, DIPEA is N,N-diisopropylethylamine, EDC is 1-[3-(dimethylaminopropyl)-3-ethylcarbodiimide, HOBt is 1-hydroxybenzotriazole and X, a, and M have the same meaning as defined previously.

Aniline A is reacted with the requisite nitrile in the presence of p-toluenesulfonic acid to afford amidine B. Amidine B is chlorinated with sodium hypochlorite and cyclized using sodium bicarbonate to form benzimidazole C. Intermediate C is saponified with sodium hydroxide to afford methoxy acid D which is reacted with various amines in the presence of HATU to afford amides F. Amides F are treated with boron tribromide to afford compounds of formula (I). Intermediate C is treated with boron tribromide to afford hydroxy acid E which is reacted with various amines using EDC and HOBt to afford compounds of formula (I).

Accordingly, the present invention provides a method for preparing the compound of the present invention, which includes the steps of:
contacting a carboxyalkyl substituted aniline derivative with a nitrile in the presence of an acid to form an intermediate amidine;
cyclizing the intermediate amidine to form a benzimidazole derivative having a carboxyalkyl;
saponifying the carboxyalkyl of the benzimidazole derivative to form a carboxylic acid; and
contacting the carboxylic acid of the benzimidazole derivative with an amine derivative, to obtain the compound of the present invention.

As used herein, the term “contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

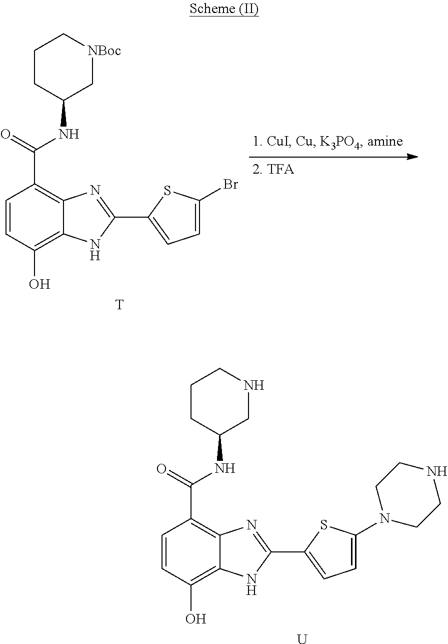

Compound T is reacted with the requisite amine in the presence of copper and copper (I) iodide followed by deprotection to afford compound U (Scheme II).

A salt, hydrate, solvate and isomer of the inventive compound of formula (I) may be prepared by employing any of the known methods. The inventive compound of formula (I), a salt, hydrate, solvate or isomer thereof may be used for the treatment of PBK dependent diseases such as cancer, by way of inhibiting PBK activity, the inventive compound having an IC₅₀ value (micro M), generally in the range of 0.0001 to 100, for example 0.001 to 50, preferably 0.001 to 10, more preferably 0.001 to 5.

Accordingly, the present invention includes a pharmaceutical composition which includes a therapeutically effective amount of the compound of formula (I), a salt, hydrate, solvate or isomer thereof as an active ingredient and a pharmaceutically acceptable carrier; therefore, the pharmaceutical composition of the present invention exerts superior preventive and treating effects on PBK dependent diseases.

A pharmaceutical formulation may be prepared in accordance with any of the conventional procedures. In preparing the formulation, the active ingredient is preferably admixed or diluted with a carrier, or enclosed within a carrier, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, calcium silicate, cellulose, methyl cellulose, microcrystalline cellulose, polyvinylpyrrolidone, water, and mineral oil. The formulations may additionally include fillers, antiemulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction.

In addition to the above, the present composition may contain other pharmaceutical active ingredients so long as they do not inhibit the in vivo function of the compound of the present invention. For example, the composition may further contain chemotherapeutic agents conventionally used for treating cancers.

The compounds disclosed herein can be used to treat or prevent PBK dependent diseases including cancer. It has been shown that PBK is a potential target for treating cancers, such as breast cancer (Example 73 of the present specification), bladder cancer (WO2006/085684), and small cell lung cancer (WO2007/013665). Accordingly, the cancer to be targeted include, but are not limited to, breast cancer, bladder cancer, and small cell lung cancer. For example, the present invention provides methods for treating or preventing PBK dependent diseases including cancer in a subject by administering to said subject the compounds disclosed herein. In a preferred embodiment, such compound can be administered to the subject in the form of pharmaceutical composition including the compound of the present invention and pharmaceutically or physiologically acceptable carrier. The pharmaceutical composition of the present invention can be administered via various routes including oral, transdermal, subcutaneous, intravenous and intramuscular introduction for treating a PBK dependent diseases including cancer in a subject.

In another embodiment, the present invention also provides the use of the compound of the present invention in manufacturing a pharmaceutical composition for treating a PBK dependent diseases including cancer. For example, the present invention relates to a use of the compound of the present invention for manufacturing a pharmaceutical composition for treating a PBK dependent diseases including cancer. In addition, the present invention further provides the compound of the present invention for use in treating a PBK dependent diseases including cancer.

Alternatively, the present invention further provides a method or process for manufacturing a pharmaceutical composition for treating PBK dependent diseases including cancer, wherein the method or process includes a step for formulating a pharmaceutically or physiologically acceptable carrier with the compound of the present invention as active ingredients.

In another embodiment, the present invention also provides a method or process for manufacturing a pharmaceutical composition for treating a PBK dependent diseases including cancer, wherein the method or process includes a step for admixing an active ingredient with a pharmaceutically or physiologically acceptable carrier, wherein the active ingredient is the compound of the present invention.

The dosage and method of administration vary according to the body-weight, age, and symptoms of the patient; however, one skilled in the art can suitably select them.

For example, although the dose of a compound of the present invention that regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering the compound parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kg of body-weight.

### EXAMPLES

The following examples are intended to further illustrate the present invention without limiting its scope.

### Example 1

### Step 1: Synthesis of Methyl 4-Methoxy-3-(thiophene-2-carboximidamido)benzoate

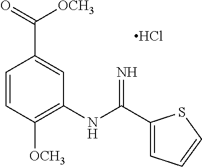

p-Toluenesulfonic acid monohydrate (42 g, 110 mmol) was heated at 120 degrees C. and once the solid completely melted, it was placed under high vacuum for 1 h to remove the water. The vacuum was released, aniline (20 g, 55 mmol) and 2-thiophenecarbonitrile (24 g, 110 mmol) were added, and the reaction mixture was heated at 160 degrees C. for 4 h. The reaction mixture was cooled to room temperature followed by addition of satd. aq NaHCO₃ (250 mL) and ethyl acetate (250 mL). The layers were separated, the aqueous layer was extracted with ethyl acetate (100 mL), and the combined organic layers were dried over Na₂SO₄, filtered, and concentrated. The crude residue was purified by column chromatography to obtain 16 g of the crude amidine intermediate. The crude intermediate was dissolved in ethyl acetate (350 mL) and HCl (2.0 M in diethyl ether, 55 mL, 110 mmol) was added. The resulting precipitate was filtered to obtain the desired product (16 g, 42% yield) as an off-white solid: ESI MS m/z 291 [C₁₄H₁₄N₃O₂S+H]⁺.

### Step 2: Synthesis of Methyl 7-Methoxy-2-(thiophen-2-yl)-1H-benzo[d]imidazole-4-carboxylate

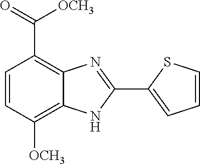

To a solution of the product from step 1 (16 g, 49 mmol) in methanol (100 mL) was added 5% aq NaOCl (75 mL, 55 mmol) and the reaction mixture was stirred at room temperature for 2 h. Next, satd. aq NaHCO₃ (150 mL) and methanol (150 mL) were added and the resulting reaction mixture was heated at 60 degrees C. for 2 d. The reaction mixture was cooled to room temperature and concentrated to remove methanol. The reaction mixture was acidified to pH 4 using 6 N HCl and the resulting precipitate was filtered and dried to obtain the desired product (8 g, 57% yield) as a brown solid: ¹H NMR (500 MHz, CDCl₃) delta 7.86 (d, J=8.5 Hz, 1H), 7.71-7.68 (m, 1H), 7.48-7.45 (m, 1H), 7.17-7.14 (m, 1H), 7.73 (d, J=8.5 Hz, 1H), 4.16 (m, 3H), 3.98 (m, 3H); ESI MS m/z 289 [C₁₄H₁₂N₂O₃S+H]⁺.

### Step 3: Synthesis of 7-Methoxy-2-(thiophen-2-yl)-1H-benzo[d]imidazole-4-carboxylic Acid

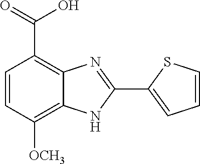

To a solution of the product from step 2 (4.2 g, 14 mmol) in ethanol (30 mL) and water (15 mL) was added 6 N NaOH (55 mL) and the reaction mixture was heated at 90 degrees C. for 2 h. The reaction mixture was cooled and concentrated to dryness. The crude residue was dissolved in water (30 ml) and acidified to pH 4 using 6 N HCl. The resulting precipitate was filtered and dried to obtain the desired product (2.2 g, 58% yield) as a brown solid: ¹H NMR (500 MHz, DMSO-d₆) delta 8.25 (d, J=3.0 Hz, 1H), 7.77 (d, J=8.0 Hz, 1H), 7.73-7.68 (m, 11-1), 7.22-7.18 (m, 1H), 6.82 (d, J=8.5 Hz, 1H), 3.97 (m, 3H); ESI MS m/z 275 [C₁₃H₁₀N₂O₃S+H]⁺.

### Step 4: Synthesis of 7-Methoxy-2-(thiophen-2-yl)-1H-benzo[d]imidazole-4-carboxylic Acid

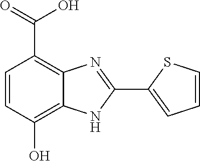

To a solution of the product from step 3 (2.5 g, 9.1 mmol) in dichloroethane (100 mL) was added BBr₃ (23 g, 91 mmol) and the reaction mixture was heated at 90 degrees C. for 2 d. The reaction mixture was cooled and poured onto ice. The resulting solids were filtered to obtain the desired product (0.45 g, 19% yield) as a brown solid. The filtrate was acidified to pH 4 using 6 N HCl and the resulting precipitate was filtered to obtain a second batch of the desired product (ALB 128328, 1.6 g, 88% yield) as a brown solid: ¹H NMR (300 MHz, CD₃OD) delta 7.93-7.90 (m, 1H), 7.75 (d, J=8.5 Hz, 1H), 7.62-7.58 (m, 1H), 7.19-7.14 (m, 1H), 6.65 (d, J=8.1 Hz, 1H); ESI MS m/z 261 [C₁₂H₈N₂O₃S+H]⁺.

### Example 2

### Step 1: Synthesis of Methyl 3-(Cyclopropanecarboximidamido)-4-methoxybenzoate Hydrochloride

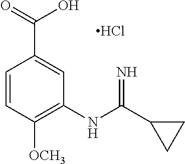

Following the procedure outlined for step 1 in Example 1, methyl 3-amino-4-methoxybenzoate (10 g, 55 mmol) was reacted with cyclopropanecarbonitrile (7.4 g, 110 mmol) to afford the desired product (16 g crude) as a black solid: ESI MS m/z 249 [C₁₃H₁₆N₂O₃+H]⁺.

### Step 2: Synthesis of Methyl 2-Cyclopropyl-7-methoxy-1H-benzo[d]imidazole-4-carboxylate

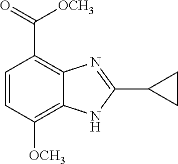

Following the procedure outlined for step 2 in Example 1, methyl 3-(cyclopropanecarboximidamido)-4-methoxybenzoate hydrochloride (15 g, 50 mmol) was reacted with aq NaOCl followed by satd. aq NaHCO₃ to afford the desired product (12 g crude) as a brown solid: ESI MS m/z 247 [C₁₃H₁₄N₂O₃+H]⁺.

### Step 3: Synthesis of 2-Cyclopropyl-7-methoxy-1H-benzo[d]imidazole-4-carboxylic Acid

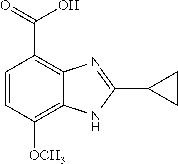

Following the procedure outlined for step 3 in Example 1, methyl 2-cyclopropyl-7-methoxy-1H-benzo[d]imidazole-4-carboxylate (2.0 g, 8.0 mmol) was reacted with sodium hydroxide to afford the desired product (1.7 g crude) as a black solid: ESI MS m/z 233 [C₁₂H₁₂N₂O₃+H]⁺.

### Step 4: Synthesis of 2-Cyclopropyl-7-hydroxy-1H-benzo[d]imidazole-4-carboxylic Acid

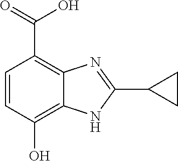

Following the procedure outlined for step 4 in Example 1, 2-cyclopropyl-7-methoxy-1H-benzo[d]imidazole-4-carboxylic acid (1.5 g, 6.1 mmol) was reacted with boron tribromide to afford the desired product (1.2 g crude) as a black solid: ESI MS m/z 219 [C₁₁H₁₀N₂O₃+H]⁺.

### Example 3

### Step 1: Synthesis of Methyl 3-(Cyclopentanecarboximidamido)-4-methoxybenzoate Hydrochloride

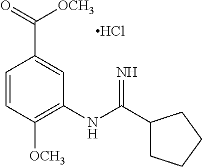

Following the procedure outlined for step 1 in Example 1, methyl 3-amino-4-methoxybenzoate (5.0 g, 27 mmol) was reacted with cyclopentanecarbonitrile (5.2 g, 55 mmol) to afford the desired product (7.7 g crude) as a brown solid: ESI MS m/z 277 [C₁₅H₂₀N₂O₃+H]⁺.

### Step 2: Synthesis of Methyl 2-Cyclopentyl-7-methoxy-1H-benzo[d]imidazole-4-carboxylate

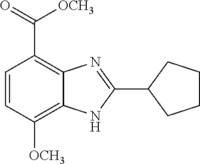

Following the procedure outlined for step 2 in Example 1, methyl 3-(cyclopentanecarboximidamido)-4-methoxybenzoate hydrochloride (5.6 g, 18 mmol) was reacted with aq NaOCl followed by satd aq NaHCO₃ to afford the desired product (4.9 g crude) as a black solid: ESI MS m/z 275 [C₁₅H₁₈N₂O₃+H]⁺.

### Step 3: Synthesis of 2-Cyclopentyl-7-hydroxy-1H-benzo[d]imidazole-4-carboxylic Acid

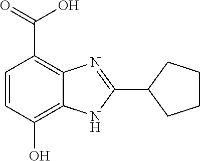

Following the procedure outlined for step 4 in Example 1, methyl 2-cyclopentyl-7-methoxy-1H-benzo[d]imidazole-4-carboxylate (1.1 g, 4.0 mmol) was reacted with boron tribromide to afford the desired product (0.92 g crude) as a black solid: ESI MS m/z 247 [C₁₃H₁₄N₂O₃+H]⁺.

### Example 4

### Step 1: Synthesis of Methyl 3-Benzimidamido-4-methoxybenzoate Hydrochloride

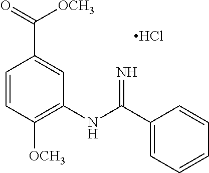

Following the procedure outlined for step 1 in Example 1, methyl 3-amino-4-methoxybenzoate (5.0 g, 27 mmol) was reacted with benzonitrile (5.7 g, 55 mmol) to afford the desired product (7.8 g crude) as a black solid: ESI MS m/z 285 [C₁₆H₁₆N₂O₃+H]⁺.

### Step 2: Synthesis of Methyl 7-Methoxy-2-phenyl-1H-benzo[d]imidazole-4-carboxylate

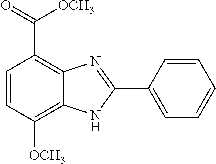

Following the procedure outlined for step 1 in Example 1, methyl 3-benzimidamido-4-methoxybenzoate hydrochloride (2.0 g, 8.0 mmol) was reacted with aq

NaOCl followed by satd aq NaHCO₃ to afford the desired product (1.7 g crude) as an off-white solid: ESI MS m/z 283 [C₁₆H₁₄N₂O₃+H]⁺.

### Step 3: Synthesis of 7-Hydroxy-2-phenyl-1H-benzo[d]imidazole-4-carboxylic Acid

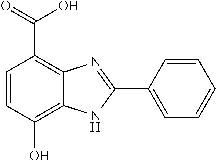

Following the procedure outlined for step 4 in Example 1, methyl 7-methoxy-2-phenyl-1H-benzo[d]imidazole-4-carboxylate (4.0 g, 12 mmol) was reacted with boron tribromide to afford the desired product (2.1 g crude) as a black solid: ESI MS m/z 255 [C₁₄H₁₀N₂O₃+H]⁺.

### General Procedure A—Synthesis of Compounds of Formula (I-II) as Described in Scheme (1):

To a solution of hydroxy acids E (1.0 equiv) in THF (5-10 mL) was added EDC (1.2 equiv), HOBt (1.1 equiv), and the amine (1.2 equiv) and the reaction mixture was either stirred at room temperature for 16 h or heated at 50-70 degrees C. for 16 h. The reaction mixture was diluted with ethyl acetate (50 mL) and washed with water (25 ml). The layers were separated and the organic layer was dried over Na₂SO₄, concentrated, and purified by preparative HPLC (C18 silica, 10-90% acetonitrile/water with 0.05% TFA). In some instances the desired product was dissolved in trifluoroacetic acid (2 mL) and stirred for 1 h at room temperature. The reaction mixture was concentrated and eluted through an ion-exchange column (using methanol and 7 N methanol in ammonia) to obtain the desired products.

### Example 5

### 2-Cyclopropyl-4-hydroxy-N-(piperidin-4-ylmethyl)-1H-benzo[d]imidazole-7-carboxamide

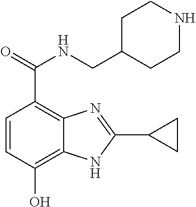

Following general procedure A, 2-cyclopropyl-4-hydroxy-1H-benzo[d]imidazole-7-carboxylic acid (55 mg, 0.25 mmol) was reacted with racemic tert-butyl 4-(aminomethyl)piperidine-1-carboxylate (81 mg, 0.38 mmol) to afford the desired product (21 mg, 27% yield) as a light brown-yellow solid: ¹H NMR (500 MHz, DMSO-d₆) delta 9.67 (bs, 1H), 7.57 (d, J=8.0 Hz, 1H), 6.58 (d, J=8.0 Hz, 1H), 3.25-3.22 (m, 2H), 2.98-2.96 (m, 2H), 2.48-2.46 (m, 2H), 2.16 (bs, 1H), 1.68-1.58 (m, 3H), 1.18-1.06 (m, 6H); ESI MS m/z 315 [C₁₇H₂₂N₄O₂+H]⁺; HPLC 98.6% (AUC), t_{R}=6.38 min.

### Example 6

### 2-Cyclopropyl-4-hydroxy-N-(piperidin-3-yl-methyl)-1H-benzo[d]imidazole-7-carboxamide

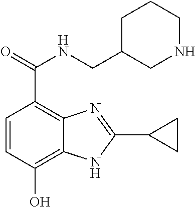

Following general procedure A, 2-cyclopropyl-4-hydroxy-1H-benzo[d]imidazole-7-carboxylic acid (55 mg, 0.25 mmol) was reacted with racemic tert-butyl 3-(aminomethyl)piperidine-1-carboxylate (81 mg, 0.38 mmol) to afford the desired product (12 mg, 15% yield) as a light gray solid: ¹H NMR (500 MHz, CD₃OD) delta 7.66 (d, J=8.0 Hz, 1H), 6.57 (d, J=8.0 Hz, 1H), 3.51-3.49 (m, 2H), 3.14 (d, J=12.5 Hz, 1H), 2.94 (bs, 1H), 2.75-2.73 (m, 1H), 2.19-2.17 (m, 1H), 1.90-1.88 (m, 2H), 1.71-1.68 (m, 2H), 1.54-1.49 (m, 1H), 1.35 (bs, 1H), 1.15-1.13 (m, 4H); ESI MS m/z 315 [C₁₇H₂₂N₄O₂+H]⁺; HPLC 99.7% (AUC), t_{R}=5.98 min.

### Example 7

### 2-Cyclopropyl-4-hydroxy-N-(piperidin-2-ylmethyl)-1H-benzo[d]imidazole-7-carboxamide

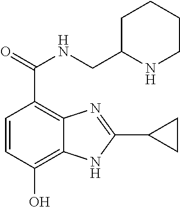

Following general procedure A, 2-cyclopropyl-4-hydroxy-1H-benzo[d]imidazole-7-carboxylic acid (55 mg, 0.25 mmol) was reacted with racemic tert-butyl 2-(aminomethyl)piperidine-1-carboxylate (81 mg, 0.38 mmol) to afford the desired product (13 mg, 17% yield) as a light gray solid: ¹H NMR (500 MHz, CD₃OD) delta 7.66 (d, J=8.0 Hz, 1H), 6.56 (d, J=8.0 Hz, 1H), 3.39-3.38 (m, 2H), 3.22 (d, J=12 Hz, 1H), 3.08 (d, J=12 Hz, 11-1), 2.69-2.68 (m, 1H), 2.66-2.63 (m, 1H), 2.55-2.50 (m, 1H), 2.18-2.15 (m, 1H), 1.99-1.97 (m, 2H), 1.92-1.89 (m, 1H), 1.82-1.80 (m, 1H), 1.62-1.59 (m, 1H), 1.32-1.34 (m, 1H), 1.13 (bs, 4H); ESI MS m/z 315 [C₁₇H₂₂N₄O₂+H]⁺; HPLC 96.8% (AUC), E_{R}=6.78 min.

### Example 8

### 2-cyclopropyl-4-hydroxy-N-(1-methylpiperidin-3-yl)-1H-benzo[d]imidazole-7-carboxamide

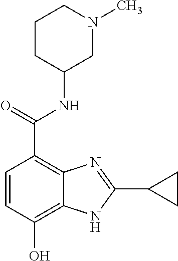

Following general procedure A, 2-cyclopropyl-4-hydroxy-1H-benzo[d]imidazole-7-carboxylic acid (55 mg, 0.25 mmol) was reacted with racemic 1-methylpiperidin-3-amine (43 mg, 0.38 mmol) to afford the desired product (21 mg, 32% yield) as a brown-yellow solid: ¹H NMR (500 MHz, DMSO-d₆) delta 12.7 (bs, 1H), 10.5 (bs, 1H), 9.97 (bs, 1H), 7.58 (d, J=8.0 Hz, 1H), 6.61 (d, J=8.0 Hz, 1H), 4.06 (bs, 1H), 3.32 (bs, 2H), 2.40-2.34 (m, 1H), 2.22-2.11 (m, 2H), 1.75 (bs, 11-0, 1.65 (bs, 1H), 1.55 (bs, 1H), 1.44 (bs, 1H), 1.14-1.10 (m, 4H); ESI MS m/z 315 [C₁₇H₂₂N₄O₂+H]⁺; HPLC 96.8% (AUC), t_{R}=7.12 min.

### Example 9

### (S)-2-cyclopropyl-4-hydroxy-N-(piperidin-3-yl)-1H-benzo[d]imidazole-7-carboxamide

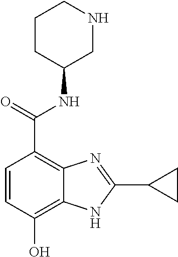

Following general procedure A, 2-cyclopropyl-4-hydroxy-1H-benzo[d]imidazole-7-carboxylic acid (55 mg, 0.25 mmol) was reacted with (S)-tert-butyl 3-aminopiperidine-1-carboxylate (75 mg, 0.38 mmol) to afford the desired product (28 mg, 37% yield) as a brown-yellow solid: ¹H NMR (500 MHz, DMSO-d₆) delta 12.7 (bs, 1H), 9.82 (bs, 1H), 7.58 (d, J=8.5 Hz, 1H), 6.61 (d, J=8.5 Hz, 1H), 3.93-3.91 (m, 1H), 3.17 (bs, 1H), 3.03-3.00 (m, 1H), 2.77 (m, 1H), 2.64 (bs, 1H), 2.16 (bs, 1H), 1.87 (bs, 1H), 1.73-1.70 (m, 1H), 1.50-1.45 (m, 2H), 1.11-1.10 (m, 4H); ESI MS m/z 301 [C₁₆H₂₀N₄O₂+H]⁺; HPLC 96.8% (AUC), t_{R}=6.63 min.

### Example 10

### 2-cyclopropyl-4-hydroxy-N-(piperidin-4-yl)-1H-benzo[d]imidazole-7-carboxamide

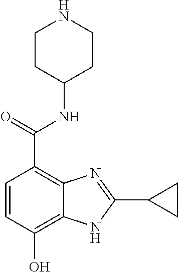

Following general procedure A, 2-cyclopropyl-4-hydroxy-1H-benzo[d]imidazole-7-carboxylic acid (55 mg, 0.25 mmol) was reacted with racemic ten-butyl 4-aminopiperidine-1-carboxylate (75 mg, 0.38 mmol) to afford the desired product (27 mg, 36% yield over two steps as a brown-yellow solid: ¹H NMR (500 MHz, DMSO-d₆) delta 9.75 (d, J=6 Hz, 1H), 7.59 (d, J=8.5 Hz, 1H), 6.61 (d, J=8.5 Hz, 1H), 3.96 (bs, 1H), 2.99-2.97 (m, 2H), 2.70-2.66 (m, 2H), 2.16 (bs, 1H), 1.88-1.86 (m, 2H), 1.42-1.40 (m, 2H), 1.13-1.04 (m, 4H); ESI MS m/z 301 [C₁₆H₂₀N₄O₂+H]⁺; HPLC 95.8% (AUC), t_{R}=6.21 min

### Example 11

### 2-cyclopropyl-4-hydroxy-N-(piperidin-3-yl)-1H-benzo[d]imidazole-7-carboxamide

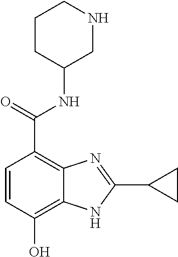

Following general procedure A, 2-cyclopropyl-4-hydroxy-1H-benzo[d]imidazole-7-carboxylic acid (55 mg, 0.25 mmol) was reacted with racemic tert-butyl 3-aminopiperidine-1-carboxylate (75 mg, 0.38 mmol) to afford the desired product (16 mg, 21% yield) as a brown-yellow solid: ¹H NMR (500 MHz, CD₃OD) delta 7.67 (d, J=8.0 Hz, 1H), 6.60 (d, J=8.0 Hz, 1H), 4.12-4.08 (m, 1H), 3.31-3.28 (m, 1H), 3.04-3.00 (m, 1H), 2.79-2.73 (m, 1H), 2.20-2.10 (m, 2H), 1.94-1.91 (m, 1H), 1.76-1.65 (m, 1H), 1.17-1.14 (m, 4H); ESI MS m/z 301 [C₁₆H₂₀N₄O₂+H]⁺; HPLC 96.8% (AUC), t_{R}=6.63 min.

### Example 12

### 2-cyclopropyl-4-hydroxy-N-(pyrrolidin-3-yl)-1H-benzo[d]imidazole-7-carboxamide

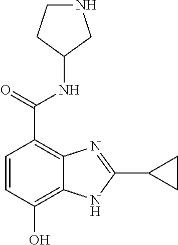

Following general procedure A, 2-cyclopropyl-4-hydroxy-1H-benzo[d]imidazole-7-carboxylic acid (55 mg, 0.25 mmol) was reacted with racemic tert-butyl 3-aminopyrrolidine-1-carboxylate (70 mg, 0.38 mmol) to afford the desired product (19 mg, 27% yield) as a brown-yellow solid: ¹H NMR (500 MHz, CD₃OD) delta 7.66 (d, J=8.0 Hz, 1H), 6.57 (d, J=8.0 Hz, 1H), 4.56-4.51 (m, 1H), 3.36-3.32 (m, 1H), 3.26-3.20 (m, 1H), 3.14-3.09 (m, 1H), 3.03-3.00 (m, 1H), 2.32-2.25 (m, 1H), 2.19-2.14 (m, 1H), 1.97-1.93 (m, 1H), 1.14-1.10 (m, 4H); ESI MS m/z 287 [C₁₅H₁₈N₄O₂+H]⁺; HPLC 96.8% (AUC), t_{R}=6.40 min.

### Example 13

### N-(azetidin-3-ylmethyl)-2-cyclopropyl-4-hydroxy-1H-benzo[d]imidazole-7-carboxamide

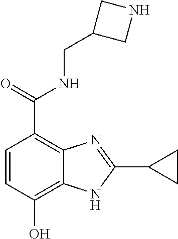

Following general procedure A, 2-cyclopropyl-4-hydroxy-1H-benzo[d]imidazole-7-carboxylic acid (55 mg, 0.25 mmol) was reacted with tert-butyl 3-(aminomethyl)azetidine-1-carboxylate (70 mg, 0.38 mmol) to afford the desired product (22 mg, 31% yield) as a brown-yellow solid: ¹H NMR (500 MHz, CD₃OD) delta 7.66 (d, J=8.5 Hz, 1H), 6.56 (d, J=8.5 Hz, 1H), 4.00-3.85 (m, 4H), 3.69-3.67 (m, 2H), 3.17-3.14 (m, 1H), 2.18-2.14 (m, 1H), 1.13-1.08 (m, 4H); ESI MS m/z 287 [C₁₅H₁₈N₄O₂+H]⁺; HPLC 96.8% (AUC), t_{R}=6.15 min.

### Example 14

### Synthesis of 2-cyclopentyl-4-hydroxy-N-(piperidin-2-ylmethyl)-1H-benzo[d]imidazole-7-carboxamide

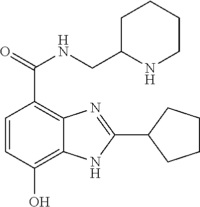

Following general procedure A, 2-cyclopentyl-4-hydroxy-1H-benzo[d]imidazole-7-carboxylic acid (62 mg, 0.25 mmol) was reacted with racemic tert-butyl 2-(aminomethyl)piperidine-1-carboxylate (81 mg, 0.38 mmol) to afford the desired product (33 mg, 39% yield) as a brown solid: ¹H NMR (300 MHz, CD₃OD) delta 7.69 (d, J=9.0 Hz, 1H), 6.59 (d, J=9.0 Hz, 1H), 3.54-3.52 (m, 2H), 3.39-3.34 (m, 1H), 3.16-3.11 (m, 1H), 2.99-2.93 (m, 1H), 2.74 (bs, 1H), 2.19-2.15 (m, 2H), 2.09-1.81 (m, 6H), 1.78-1.69 (m, 3H), 1.52-1.32 (m, 3H); ESI MS m/z 343 [C₁₉H₂₆N₄O₂+H]⁺; HPLC 98.6% (AUC), t_{R}=1.51 min.

### Example 15

### 2-Cyclopentyl-4-hydroxy-N-(piperidin-3-ylmethyl)-1H-benzo[d]imidazole-7-carboxamide

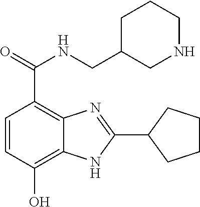

Following general procedure A, 2-cyclopentyl-4-hydroxy-1H-benzo[d]imidazole-7-carboxylic acid (62 mg, 0.25 mmol) was reacted with racemic tert-butyl 3-(aminomethyl)piperidine-1-carboxylate (81 mg, 0.38 mmol) to afford the desired product (35 mg, 41% yield) as a off-white solid: ¹H NMR (300 MHz, CD₃OD) delta 7.69 (d, J=9.0 Hz, 1H), 6.59 (d, J=9.0 Hz, 1H), 3.43-3.41 (m, 2H), 3.30-3.25 (m, 1H), 3.16-3.12 (m, 1H), 2.72-2.59 (m, 2H), 2.18-2.15 (m, 2H), 2.03-1.75 (m, 11H), 1.39-1.34 (m, 1H); ESI MS m/z 343 [C₁₉H₂₆N₄O₂+H]⁺; HPLC 99.2% (AUC), t_{R}=1.49 min.

### Example 16

### (S)-2-Cyclopentyl-4-hydroxy-N-(piperidin-3-yl)-1H-benzo[d]imidazole-7-carboxamide

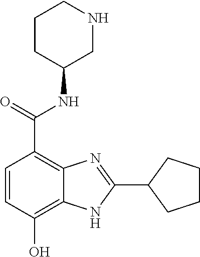

Following general procedure A, 2-cyclopentyl-4-hydroxy-1H-benzo[d]imidazole-7-carboxylic acid (55 mg, 0.25 mmol) was reacted with (S)-tert-butyl 3-aminopiperidine-1-carboxylate (75 mg, 0.38 mmol) to afford the desired product (22 mg, 27% yield) as a brown solid: ¹H NMR (500 MHz, CD₃OD) delta 7.69 (d, J=8.0 Hz, 1H), 6.61 (d, J=8.0 Hz, 1H), 4.09 (bs, 1H), 3.39-3.30 (m, 2H), 2.99 (bs, 1H), 2.72-2.76 (m, 2H), 2.19-2.14 (m, 3H), 2.03-1.99 (m, 2H), 1.91-1.88 (m, 3H), 1.78-1.67 (m, 4H); ESI MS m/z 329 [C₁₈H₂₄N₄O₂+H]⁺; HPLC >99% (AUC), t_{R}=1.48 min.

### Example 17

### (S)-4-Hydroxy-2-phenyl-N-(piperidin-3-yl)-1H-benzo[d]imidazole-7-carboxamide

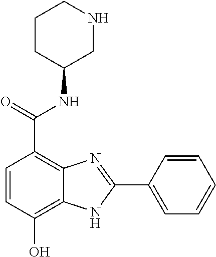

Following general procedure A, 4-hydroxy-2-phenyl-1H-benzo[d]imidazole-7-carboxylic acid (55 mg, 0.25 mmol) was reacted with (S)-tert-butyl 3-aminopiperidine-1-carboxylate (75 mg, 0.38 mmol) to afford the desired product (10 mg, 12% yield) as a green-yellow solid: ¹H NMR (500 MHz, CD₃OD) delta 8.20-8.18 (m, 2H), 7.78 (d, J=8.5 Hz, 1H), 7.56-7.51 (m, 3H), 6.68 (d, J=8.5 Hz, 1H), 4.16 (bs, 1H), 3.39-3.35 (m, 1H), 3.05 (bs, 1H), 2.87-2.82 (m, 2H), 2.20-2.19 (m, 1H), 2.00 (bs, 1H), 1.80-1.76 (m, 2H); ESI MS m/z 337 [C₁₉H₂₀N₄O₂+H]⁺; HPLC 95.7% (AUC), t_{R}=8.78 min.

### Example 18

### 4-Hydroxy-2-phenyl-N-(piperidin-2-ylmethyl)-1H-benzo[d]imidazole-7-carboxamide

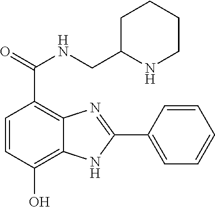

Following general procedure A, 4-hydroxy-2-phenyl-1H-benzo[d]imidazole-7-carboxylic acid (62 mg, 0.25 mmol) was reacted with racemic tert-butyl 2-(aminomethyl)piperidine-1-carboxylate (81 mg, 0.38 mmol) to afford the desired product (20 mg, 23% yield) as a brown solid: ¹H NMR (500 MHz, DMSO-d₆) delta 9.77 (bs, 1H), 8.32-8.30 (m, 2H), 7.70 (d, J=8.5 Hz, 1H), 7.58-7.51 (m, 3H), 6.71 (d, J=8.5 Hz, 1H), 3.17-3.10 (m, 2H), 2.96-2.94 (m, 1H), 2.56-2.42 (m, 3H), 1.90-1.87 (m, 1H), 1.81-1.77 (m, 1H), 1.69-1.65 (m, 1H), 1.46-1.44 (m, 1H), 1.27-1.24 (m, 1H); ESI MS m/z 351 [C₂₀H₂₂N₄O₂+H]⁺; HPLC 99.0% (AUC), t_{R}=7.74 min.

### Example 19

### 4-hydroxy-2-phenyl-N-(piperidin-3-ylmethyl)-1H-benzo[d]imidazole-7-carboxamide

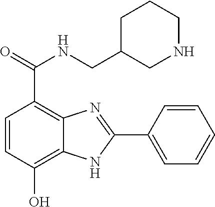

Following general procedure A, 4-hydroxy-2-phenyl-1H-benzo[d]imidazole-7-carboxylic acid (62 mg, 0.25 mmol) was reacted with tert-butyl 3-(aminomethyl)piperidine-1-carboxylate (81 mg, 0.38 mmol) to afford the desired product (20 mg, 23% yield) as a brown solid: ¹H NMR (500 MHz, DMSO-d₆) delta 9.99 (bs, 1H), 8.40-8.38 (m, 2H), 7.70 (d, J=8.5 Hz, 2H), 7.57-7.52 (m, 3H), 6.72 (d, J=8.5 Hz, 1H), 3.43-3.41 (m, 2H), 3.11-3.08 (m, 1H), 2.64 (bs, 1H), 1.78-1.71 (m, 2H), 1.57 (bs, 1H), 1.69-1.65 (m, 1H), 1.46-1.44 (m, 1H), 1.27-1.24 (m, 1H); ESI MS m/z 351 [C₂₀H₂₂N₄O₂+H]⁺; HPLC 99.1 (AUC), t_{R}=8.99 min.

### Example 20

### 7-Hydroxy-N-(4-hydroxycyclohexyl)-2-(thiophen-2-yl)-1H-benzo[d]imidazole-4-carboxamide

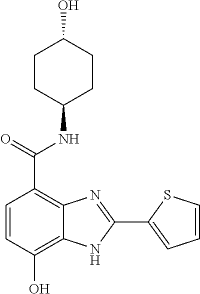

To a solution of 4-hydroxy-2-(thiophen-2-yl)-1H-benzo[d]imidazole-7-carboxylic acid (0.15 g, 0.57 mmol) in DMF (10 mL) was added HATU (0.26 g, 0.68 mmol), DIPEA (0.30 mL, 1.7 mmol) and trans-4-aminocyclohexanol (0.13 g, 1.1 mmol). The reaction mixture was heated at 50 degrees C. for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (13 mg, 32%) as an off-white solid: ¹H NMR (300 MHz, CD₃OD) delta 10.19-10.17 (m, 1H), 7.87-7.85 (m, 1H), 7.79-7.75 (m, 1H), 7.64-7.61 (m, 1H), 7.22-7.19 (m, 1H), 6.71-6.67 (m, 1H), 4.02-3.97 (m, 1H), 3.77-3.71 (m, 1H), 2.19-2.08 (m, 4H), 1.55-1.50 (m, 4H); ESI MS m/z 358 [C₁₈H₁₉N₃O₃S+H]⁺; HPLC 98.8% (AUC), t_{R}=11.84 min.

### Example 21

### (7-hydroxy-2-[thiophen-2-yl]-1H-benzo[d]imidazol-4-yl) (piperazin-1-yl)methanone

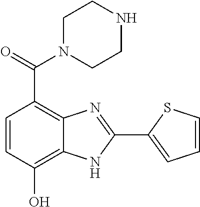

To a solution of 4-hydroxy-2-(thiophen-2-yl)-1H-benzo[d]imidazole-7-carboxylic acid (0.20 g, 0.76 mmol) in DMF (10 mL) was added HATU (0.34 g, 0.92 mmol), DIPEA (0.39 mL, 2.3 mmol) and tert-butyl piperazine-1-carboxylate (0.17 g, 0.92 mmol). The reaction mixture was heated at 50 degrees C. for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The intermediate was dissolved in methylene dichloride and treated with 2 N HCl in ether and the reaction mixture was stirred at room temperature for 6 h. The reaction mixture was concentrated and the residue was eluted through an ion-exchange column (using methanol and 7 N methanol in ammonia) to afford the desired product (13 mg, 32%) as an off-white solid: ¹H NMR (500 MHz, DMSO-d₆) delta 8.01 (bs, 1H), 7.70 (d, J=5.0, Hz, 1H), 7.20 (dd, J=5.0, 4.0 Hz, 1H), 7.00 (d, J=8.0, Hz, 1H), 6.56-6.57 (m, 1H), 3.70-3.05 (m, 8H); ESI MS m/z 329 [C₁₆H₁₆N₄O₂S+H]⁺; HPLC 95.5% (AUC), t_{R}=8.79 min.

General Procedure B—Synthesis of Amides F as Described in Scheme (1):

To a suspension of 7-methoxy-2-(thiophen-2-yl)-1H-benzo[d]imidazole-4-carboxylic acid (1.0 equiv) in toluene (5-15 mL) was added thionyl chloride (4.0 equiv). After stirring at room temperature for 16 h, the reaction mixture was heated at 70 degrees C. for 2 h. The reaction mixture was cooled, and concentrated, and the residue was suspended in THF (10-20 mL) followed by the addition of pyridine (2.0 equiv) and the corresponding amine (2.0 equiv) and the reaction mixture was heated at 70 degrees C. for 16 h. The reaction mixture was concentrated and the residue was diluted with water (20 mL) and extracted with ethyl acetate (3×20 mL). The combined organic layers were washed with satd. aq NaHCO₃ (20 mL), concentrated, and purified by flash chromatography (silica, 0-15% methanol/dichloromethane) to afford amides F. In most cases these intermediates were isolated as crude products and were carried forward without extensive characterization or further purification.

### Example 22

### tert-Butyl 3-[7-methoxy-2-(thiophen-2-yl)-1H-benzo[d]imidazole-4-carboxamido]piperidine-1-carboxylate

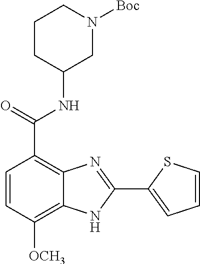

Following general procedure B, 4-methoxy-2-(thiophen-2-yl)-1H-benzo[d]imidazole-7-carboxylic acid (0.15 g, 0.44 mmol) was reacted with racemic 3-amino-1-boc-piperidne (0.18 g, 0.88 mmol) to afford the desired product (0.13 g) as a brown solid: ESI MS m/z 443 [C₂₃H₂₈N₄O₄S+H]⁺.

### Example 23

### tert-Butyl 4-{2-[7-methoxy-2-(thiophen-2-yl)-1H-benzo[d]imidazole-4-carboxamido]ethyl}piperazine-1-carboxylate

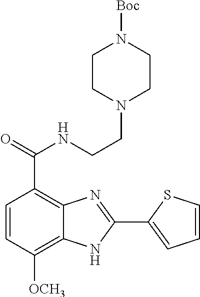

Following general procedure B, 4-methoxy-2-(thiophen-2-yl)-1H-benzo[d]imidazole-7-carboxylic acid (0.16 g, 0.58 mmol) was reacted with tert-butyl 4-(2-aminoethyl)piperazine-1-carboxylate (0.27 g, 1.2 mmol) to afford the desired product (0.24 g) as a foam: ESI MS m/z 486 [C₂₄H₃₁N₅O₄S+H]⁺.

### Example 24

### (R)-tert-Butyl 3-[7-methoxy-2-(thiophen-2-yl)-1H-benzo[d]imidazole-4-carboxamido]piperidine-1-carboxylate

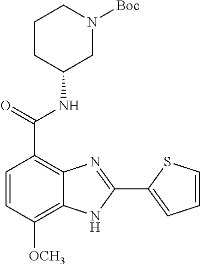

Following general procedure B, 4-methoxy-2-(thiophen-2-yl)-1H-benzo[d]imidazole-7-carboxylic acid (0.13 g, 0.46 mmol) was reacted with (R)-3-amino-1-boc-piperidine (0.18 g, 0.92 mmol) to afford the desired product (0.12 g) as a brown solid: ESI MS m/z 457 [C₂₃H₂₈N₄O₄S+H]⁺.

### Example 25

### (S)-tert-Butyl 3-[7-methoxy-2-(thiophen-2-yl)-1H-benzo[d]imidazole-4-carboxamido]piperidine-1-carboxylate

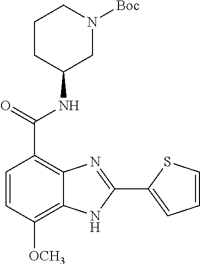

Following general procedure B, 4-methoxy-2-(thiophen-2-yl)-1H-benzo[d]imidazole-7-carboxylic acid (0.13 g, 0.46 mmol) was reacted with (S)-3-amino-1-boc-piperidine (0.18 g, 0.92 mmol) to afford the desired product (0.13 g) as a brown oil: ESI MS m/z 457 [C₂₃H₂₈N₄O₄S+H]⁺.

### Example 26

### tert-Butyl 3-{[7-methoxy-2-(thiophen-2-yl)-1H-benzo[d]imidazole-4-carboxamido]methyl}piperidine-1-carboxylate

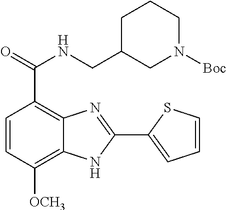

Following general procedure B, 4-methoxy-2-(thiophen-2-yl)-1H-benzo[d]imidazole-7-carboxylic acid (0.17 g, 0.62 mmol) was reacted with racemic 3-aminomethyl-1-boc-piperidine (0.26 g, 1.2 mmol) to afford the desired product (0.23 g) as a brown oil: ESI MS m/z 471 [C₂₄H₃₀N₄O₄S+H]⁺.

### Example 27

### tert-Butyl 4-[7-methoxy-2-(thiophen-2-yl)-1H-benzo[d]imidazole-4-carboxamido]piperidine-1-carboxylate

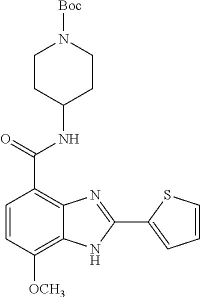

Following general procedure B, 4-methoxy-2-(thiophen-2-yl)-1H-benzo[d]imidazole-7-carboxylic acid (0.16 g, 0.58 mmol) was reacted with 4-amino-1-boc-piperidine (0.23 g, 1.2 mmol) to afford the desired product (0.20 g) as a brown oil: ESI MS m/z 457 [C₂₃H₂₈N₄O₄S+H]⁺.

### Example 28

### 7-Methoxy-N-(1-methylpiperidin-3-yl)-2-(thiophen-2-yl)-1H-benzo[d]imidazole-4-carboxamide

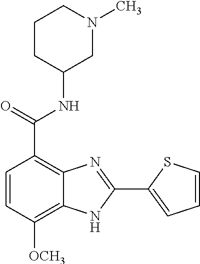

Following general procedure B, 4-methoxy-2-(thiophen-2-yl)-1H-benzo[d]imidazole-7-carboxylic acid (0.16 g, 0.59 mmol) was reacted with racemic 1-methyl-piperidin-3-amine (0.14 g, 1.2 mmol) to afford the desired product (0.15 g) as a brown glass: ESI MS m/z 371 [C₁₉H₂₂H₄O₂S+H]⁺.

### Example 29

### tert-Butyl 4-{[7-methoxy-2-(thiophen-2-yl)-1H-benzo[d]imidazole-4-carboxamido]methyl}piperidine-1-carboxylate

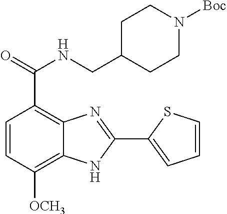

Following general procedure B, 4-methoxy-2-(thiophen-2-yl)-1H-benzo[d]imidazole-7-carboxylic acid (0.15 g, 0.56 mmol) was reacted with 4-aminomethyl-1-boc-piperidine (0.24 g, 1.1 mmol) to afford the desired product (0.16 g) as a brown foam: ESI MS m/z 471 [C₂₄H₃₀N₄O₄S+H]⁺.

### Example 30

### tert-Butyl 3-{[7-methoxy-2-(thiophen-2-yl)-1H-benzo[d]imidazole-4-carboxamido]methyl}azetidine-1-carboxylate

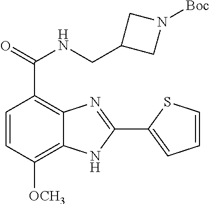

Following general procedure B, 4-methoxy-2-(thiophen-2-yl)-1H-benzo[d]imidazole-7-carboxylic acid (0.15 g, 0.56 mmol) was reacted with 1-boc-3(aminomethyl)azetidine (0.20 g, 1.1 mmol) to afford the desired product (0.17 g) as a brown foam: ESI MS m/z 443 [C₂₂H₂₆N₄O₄S+H]⁺.

### Example 31

### tert-Butyl 3-[7-methoxy-2-(thiophen-2-yl)-1H-benzo[d]imidazole-4-carboxamido]pyrrolidine-1-carboxylate

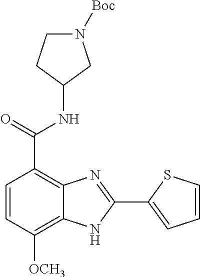

Following general procedure B, 4-methoxy-2-(thiophen-2-yl)-1H-benzo[d]imidazole-7-carboxylic acid (0.15 g, 0.56 mmol) was reacted with 3-amino-1-Boc-pyrrolidine (0.21 g, 1.1 mmol) to afford the desired product (0.20 g) as a brown oil: ESI MS m/z 443 [C₂₂H₂₆N₄O₄S+H]⁺.

### Example 32

### tert-Butyl 2-{[7-methoxy-2-(thiophen-2-yl)-1H-benzo[d]imidazole-4-carboxamido]methyl}piperidine-1-carboxylate

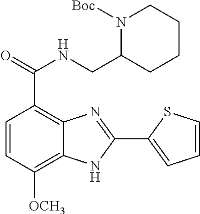

Following general procedure B, 4-methoxy-2-(thiophen-2-yl)-1H-benzo[d]imidazole-7-carboxylic acid (0.16 g, 0.58 mmol) was reacted with racemic 2-(aminomethyl)-1-N-boc-piperidine (0.25 g, 1.2 mmol) to afford the desired product (0.23 g) as a brown foam: ESI MS m/z 471 [C₂₄H₃₀N₄O₄S+H]⁺.

### Example 33

### tert-Butyl 3-{[7-methoxy-2-(thiophen-2-yl)-1H-benzo[d]imidazole-4-carboxamido]methyl}pyrrolidine-1-carboxylate

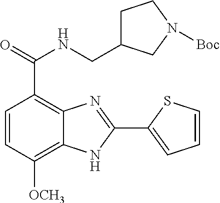

Following general procedure B, 4-methoxy-2-(thiophen-2-yl)-1H-benzo[d]imidazole-7-carboxylic acid (0.16 g, 0.58 mmol) was reacted with 3-(aminomethyl)-1-N-Boc-pyrrolidine (0.24 g, 1.2 mmol) to afford the desired product (0.19 g) as a brown oil: ESI MS m/z 457 [C₂₃H₂₈N₄O₄S+H]⁺.

### Example 34

### tert-Butyl-4-[7-methoxy-2-(thiophen-2-yl)-1H-benzo[d]imidazole-4-carboxamido]cyclohexylcarbamate

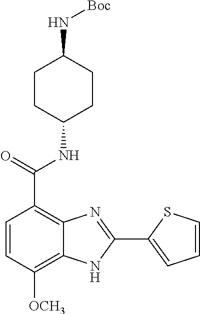

Following general procedure B, 4-methoxy-2-(thiophen-2-yl)-1H-benzo[d]imidazole-7-carboxylic acid (0.15 g, 0.55 mmol) was reacted with 1-Boc-amino-1,4-cyclohexyldiamine (0.23 g, 1.1 mmol) to afford the desired product (92 mg) as a brown oil: ESI MS m/z 471 [C₂₄H₃₀N₄O₄S+H]⁺.

### General Procedure C—Synthesis of Compounds as Described in Scheme (1):

To a suspension of amides F (1.0 equiv) in dichloroethane (10-25 mL) was added boron tribromide (6.0-10 equiv) and the reaction mixture was heated at 80 degrees C. for 16 h. The reaction mixture was poured over ice and the resulting mixture was concentrated. The crude residue was eluted through an ion-exchange column (using methanol and 7 N methanol in ammonia) as a crude purification. The crude product was further purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products.

### Example 35

### 7-Hydroxy-N-(piperidin-3-yl)-2-(thiophen-2-yl)-1H-benzo[d]imidazole-4-carboxamide

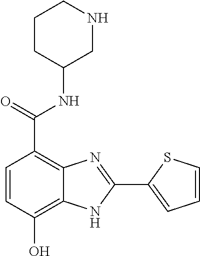

Following general procedure C, tert-Butyl 3-[7-methoxy-2-(thiophen-2-yl)-1H-benzo[d]imidazole-4-carboxamido]piperidine-1-carboxylate (0.13 g) was reacted with boron tribromide to afford the desired product (34 mg, 23% yield) as a light yellow solid: ¹H NMR (300 MHz, CD₃OD) delta 7.86-7.85 (m, 1H), 7.76 (d, J=8.4 Hz, 1H), 7.63-7.61 (m, 1H), 7.22-7.19 (m, 1H), 6.66 (d, J=8.4 Hz, 1H), 4.14-4.10 (m, 1H), 3.04-3.00 (m, 1H), 2.86-2.77 (m, 2H), 2.18-1.99 (m, 2H), 1.79-1.72 (m, 2H); ESI MS m/z 343 [C₁₇H₁₈N₄O₂S+H]⁺; HPLC 99.2% (AUC), t_{R}=9.73 min.

### Example 36

### 7-Hydroxy-N-[2-(piperazin-1-yl)ethyl]-2-(thiophen-2-yl)-1H-benzo[d]imidazole-4-carboxamide

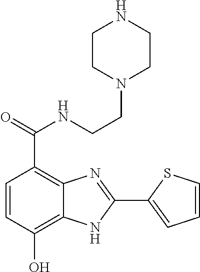

Following general procedure C, tert-Butyl

4-{2-[7-methoxy-2-(thiophen-2-yl)-1H-benzo[d]imidazole-4-carboxamido]ethyl}piperazine-1-carboxylate (0.24 g) was reacted with boron tribromide to afford the desired product (70 mg, 32% yield) as a white solid: ¹H NMR (500 MHz, DMSO-d₆) delta 9.50 (s, 1H), 8.08 (d, J=2.0 Hz, 1H), 7.77 (d, J=5.0 Hz, 1H), 7.69 (d, J=8.0 Hz, 1H), 7.25-7.24 (m, 1H), 6.71 (d, J=8.0 Hz, 1H), 3.55-3.51 (m, 3H), 2.90-2.84 (m, 5H), 2.56-2.50 (m, 3H); ESI MS m/z 372 [C₁₈H₂₁N₅O₂S+H]⁺; HPLC >99% (AUC), t_{R}=8.74 min.

### Example 37

### (R)-7-Hydroxy-N-(piperidin-3-yl)-2-(thiophen-2-yl)-1H-benzo[d]imidazole-4-carboxamide

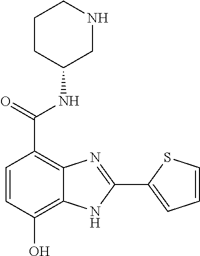

Following general procedure C, (R)-tert-Butyl 3-[7-methoxy-2-(thiophen-2-yl)-1H-benzo[d]imidazole-4-carboxamido]piperidine-1-carboxylate (0.12 g) was reacted with boron tribromide to afford the desired product (25 mg, 16% yield) as a light yellow solid: ¹H NMR (300 MHz, CD₃OD) delta 7.88-7.87 (m, 1H), 7.79-7.75 (m, 1H), 7.65-7.63 (m, 1H), 7.24-7.21 (m, 1H), 6.70-6.67 (m, 1H), 4.17-4.14 (m, 1H), 3.08-3.00 (m, 1H), 2.89-2.78 (m, 2H), 2.24-1.98 (m, 2H), 1.82-1.76 (m, 2H); ESI MS m/z 343 [C₁₇H₁₈N₄O₂S+H]⁺; HPLC 96.1% (AUC), t_{R}=10.50 min.

### Example 38

### (S)-7-Hydroxy-N-(piperidin-3-yl)-2-(thiophen-2-yl)-1H-benzo[d]imidazole-4-carboxamide

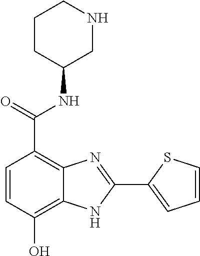

Following general procedure C, (S)-tert-Butyl 3-[7-methoxy-2-(thiophen-2-yl)-1H-benzo[d]imidazole-4-carboxamido]piperidine-1-carboxylate (0.13 g) was reacted with boron tribromide to afford the desired product (45 mg, 29% yield) as a light yellow solid: ¹H NMR (300 MHz, CD₃OD) delta 7.88-7.87 (m, 1H), 7.79-7.75 (m, 1H), 7.65-7.63 (m, 1H), 7.24-7.21 (m, 1H), 6.70-6.66 (m, 1H), 4.17-4.14 (m, 1H), 3.08-3.00 (m, 1H), 2.89-2.78 (m, 2H), 2.24-1.98 (m, 2H), 1.82-1.76 (m, 2H); ESI MS m/z 343 [C₁₇H₁₈N₄O₂S+H]⁺; HPLC >99% (AUC), t_{R}=9.80 min.

### Example 39

### 7-Hydroxy-N-(piperidin-3-ylmethyl)-2-(thiophen-2-yl)-1H-benzo[d]imidazole-4-carboxamide

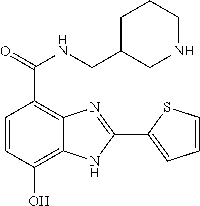

Following general procedure C, tert-Butyl 3-{[7-methoxy-2-(thiophen-2-yl)-1H-benzo[d]imidazole-4-carboxamido]methyl}piperidine-1-carboxylate (0.23 g) was reacted with boron tribromide to afford the desired product (90 mg, 41% yield) as a light brown solid: ¹H NMR (300 MHz, DMSO-d₆) delta 9.62 (s, 1H), 8.06-8.04 (m, 1H), 7.74 (d, J=4.8 Hz, 1H), 7.64 (d, J=8.4 Hz, 1H), 7.24-7.21 (m, 1H), 6.66 (d, J=8.4 Hz, 1H), 3.29 (t, J=6.0 Hz, 2H), 3.17-3.10 (m, 1H), 2.93-2.89 (m, 1H), 2.47-2.37 (m, 2H), 1.95-1.90 (m, 1H), 1.76-1.63 (m, 2H), 1.49-1.20 (m, 2H); ESI MS m/z 357 [C₁₈H₂₀N₄O₂S+H]⁺; HPLC >99% (AUC), t_{R}=9.41 min.

### Example 40

### 7-Hydroxy-N-(piperidin-4-yl)-2-(thiophen-2-yl)-1H-benzo[d]imidazole-4-carboxamide

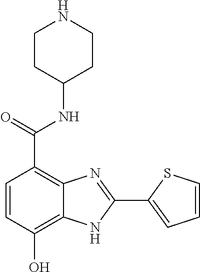

Following general procedure C, tert-Butyl 4-[7-methoxy-2-(thiophen-2-yl)-1H-benzo[d]imidazole-4-carboxamido]piperidine-1-carboxylate (0.2 g) was reacted with boron tribromide to afford the desired product (85 mg, 42% yield) as a light yellow solid: ¹H NMR (300 MHz, CD₃OD) delta 7.85-7.84 (m, 1H), 7.76 (d, J=5.1 Hz, 1H), 7.62-7.60 (m, 1H), 7.21-7.19 (m, 1H), 6.66 (d, J=5.1 Hz, 1H), 4.21-4.20 (m, 1H), 3.29-3.24 (m, 2H), 2.99-2.93 (m, 2H), 2.17-2.14 (m, 2H), 1.80-1.74 (m, 2H); ESI MS m/z 343 [C₁₇H₁₈N₄O₂S+H]⁺; HPLC >99% (AUC), t_{R}=9.07 min.

### Example 41

### 7-Hydroxy-N-(1-methylpiperidin-3-yl)-2-(thiophen-2-yl)-1H-benzo[d]imidazole-4-carboxamide

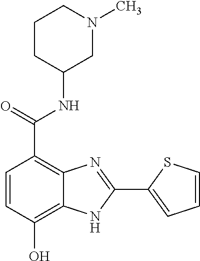

Following general procedure C, 7-methoxy-N-(1-methylpiperidin-3-yl)-2-(thiophen-2-yl)-1H-benzo[d]imidazole-4-carboxamide (0.15 g) was reacted with boron tribromide to afford the desired product (75 mg, 36% yield) as a light yellow solid: ¹H NMR (300 MHz, CD₃OD) delta 7.89-7.88 (m, 1H), 7.78 (d, J=8.4 Hz, 1H), 7.65-7.64 (m, 1H), 7.23-7.20 (m, 1H), 6.71 (d, J=8.4 Hz, 1H), 4.26-4.24 (m, 1H), 3.01-2.98 (m, 1H), 2.67-2.65 (m, 1H), 2.38 (s, 5H), 2.05-1.92 (m, 2H), 1.80-1.59 (m, 2H); ESI MS m/z 357 [C₁₈H₂₀N₄O₂S+H]⁺; HPLC 96.2% (AUC), t_{R}=9.55 min.

### Example 42

### 7-Hydroxy-N-(piperidin-4-ylmethyl)-2-(thiophen-2-yl)-1H-benzo[d]imidazole-4-carboxamide

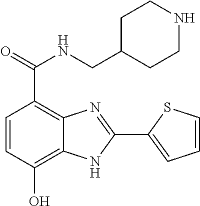

Following general procedure C, tert-Butyl 4-{[7-methoxy-2-(thiophen-2-yl)-1H-benzo[d]imidazole-4-carboxamido]methyl}piperidine-1-carboxylate (0.16 g) was reacted with boron tribromide to afford the desired product (700 mg, 35% yield) as a yellow solid: ¹H NMR (300 MHz, CD₃OD) delta 7.85-7.84 (m, 1H), 7.78-7.74 (m, 1H), 7.63-7.61 (m, 1H), 7.23-7.19 (m, 1H), 6.64-6.61 (m, 1H), 3.49 (d, J=6.6 Hz, 2H), 2.88-2.79 (m, 2H), 2.07-2.03 (m, 2H), 1.94-1.93 (m, 1H), 1.56-1.44 (m, 2H); ESI MS m/z 357 [C₁₈H₂₀N₄O₂S+H]⁺; HPLC >99% (AUC), t_{R}=9.15 min.

### Example 43

### N-(Azetidin-3-ylmethyl)-7-hydroxy-2-(thiophen-2-yl)-1H-benzo[d]imidazole-4-carboxamide

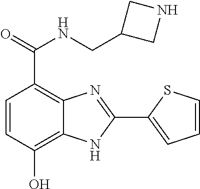

Following general procedure C, tert-Butyl 3-{[7-methoxy-2-(thiophen-2-yl)-1H-benzo[d]imidazole-4-carboxamido]methyl}azetidine-1-carboxylate (0.17 g) was reacted with boron tribromide to afford the desired product (43 mg, 24% yield) as a light yellow solid: ¹H NMR (300 MHz, CD₃OD) delta 7.84-7.83 (m, 1H), 7.74 (d, J=8.4 Hz, 1H), 7.62-7.59 (m, 1H), 7.22-7.19 (m, 1H), 6.61 (d, J=8.4 Hz, 1H), 4.02-3.96 (m, 2H), 3.90-2.84 (m, 2H), 3.74 (d, J=6.3 Hz, 2H); ESI MS m/z 329 [C₁₆H₁₆N₄O₂S+H]⁺; HPLC >99% (AUC), t_{R}=8.70 min.

### Example 44

### 7-Hydroxy-N-(pyrrolidin-3-yl)-2-(thiophen-2-yl)-1H-benzo[d]imidazole-4-carboxamide

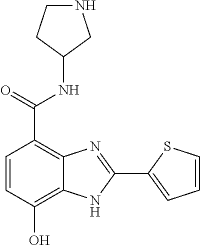

Following general procedure C, tert-Butyl 3-[7-methoxy-2-(thiophen-2-yl)-1H-benzo[d]imidazole-4-carboxamido]pyrrolidine-1-carboxylate (0.20 g) was reacted with boron tribromide to afford the desired product (0.12 g, 63% yield) as a light brown solid: ¹H NMR (300 MHz, CD₃OD) delta 8.09 (s, 1H), 7.90 (d, J=8.4 Hz, 2H), 7.36-7.33 (m, 1H), 6.87 (d, J=8.4 Hz, 1H), 4.75-4.71 (m, 1H), 3.69-3.64 (m, 2H), 3.54-3.48 (m, 2H), 2.54-2.50 (m, 1H), 2.35-2.30 (m, 1H); ESI MS m/z 329 [C₁₆H₁₆N₄O₂S+H]⁺; HPLC >99% (AUC), t_{R}=8.80 min.

### Example 45

### 7-Hydroxy-N-(piperidin-2-ylmethyl)-2-(thiophen-2-yl)-1H-benzo[d]imidazole-4-carboxamide

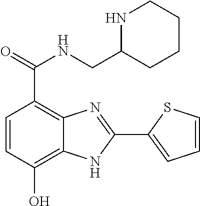

Following general procedure C, tert-Butyl 2-{[7-methoxy-2-(thiophen-2-yl)-1H-benzo[d]imidazole-4-carboxamido]methyl}piperidine-1-carboxylate (0.23 g) was reacted with boron tribromide to afford the desired product (90 mg, 44% yield) as a yellow solid: ¹H NMR (300 MHz, CD₃OD) delta 8.03-8.02 (m, 1H), 7.87 (d, J=8.4

Hz, 1H), 7.82-7.81 (m, 1H), 7.32-7.29 (m, 1H), 6.83 (d, J=8.4 Hz, 1H), 3.78-3.75 (m, 2H), 3.44-3.36 (m, 2H), 3.06-3.02 (m, 1H), 2.14-2.10 (m, 1H), 2.00-1.90 (m, 2H), 1.75-1.66 (m, 3H); ESI MS m/z 357 [C₁₈H₂₀N₄O₂S+H]⁺; HPLC >99% (AUC), t_{R}=9.49 min.

### Example 46

### 7-Hydroxy-N-(pyrrolidin-3-ylmethyl)-2-(thiophen-2-yl)-1H-benzo[d]imidazole-4-carboxamide

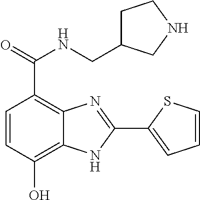

Following general procedure C, tert-Butyl 3-{[7-methoxy-2-(thiophen-2-yl)-1H-benzo[d]imidazole-4-carboxamido]methyl}pyrrolidine-1-carboxylate (0.19 g) was reacted with boron tribromide to afford the desired product (79 mg, 39% yield) as a light yellow solid: ¹H NMR (300 MHz, CD₃OD) delta 7.84-7.82 (m, 1H), 7.75 (d, J=8.4 Hz, 1H), 7.61-7.59 (m, 1H), 7.21-7.18 (m, 1H), 6.61 (d, J=8.4 Hz, 1H), 3.63-3.54 (m, 2H), 3.37-3.33 (m, 1H), 3.27-3.06 (m, 2H), 2.98-2.91 (m, 1H), 2.66-2.61 (m, 1H), 2.24-2.18 (m, 1H), 1.86-1.79 (m, 1H); ESI MS m/z 343 [C₁₇H₁₈N₄O₂S+H]⁺; HPLC >99% (AUC), t_{R}=8.91 min.

### Example 47

### N-(4-Aminocyclohexyl)-7-hydroxy-2-(thiophen-2-yl)-1H-benzo[d]imidazole-4-carboxamide

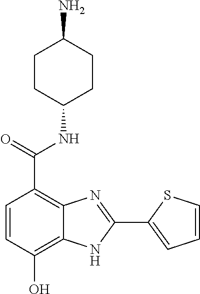

Following general procedure C, tert-Butyl-4-[7-methoxy-2-(thiophen-2-yl)-1H-benzo[d]imidazole-4-carboxamido]cyclohexylcarbamate (92 mg) was reacted with boron tribromide to afford the desired product (21 mg, 10% yield over two steps) as a light yellow solid: ¹H NMR (300 MHz, CD₃OD) delta 7.85-7.84 (m, 1H), 7.77 (d, J=8.4 Hz, 1H), 7.61-7.59 (m, 1H), 7.22-7.17 (m, 1H), 6.63 (d, J=8.4 Hz, 1H), 4.24-4.23 (m, 1H), 3.01-2.97 (m, 1H), 2.15-2.10 (m, 2H), 2.03-1.78 (m, 6H); ESI MS m/z 357 [C₁₈H₂₀N₄O₂S+H]⁺; HPLC 95.6% (AUC), t_{R}=9.22 min.

### Example 48

### 2-(Bicyclo[2.2.1]heptan-2-yl)-7-hydroxy-N-(piperidin-3-ylmethyl)-1H-benzo[d]imidazole-4-carboxamide

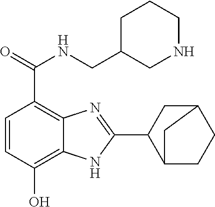

Following General Procedure C, tert-Butyl 3-((2-(bicyclo[2.2.1]heptan-2-yl)-7-methoxy-1H-benzo[d]imidazole-4-carboxamido)methyl)piperidine-1-carboxylate (330 mg crude) was reacted with boron tribromide to afford the desired product (71 mg, 45% yield) as a light yellow solid: ¹H NMR (500 MHz, CD₃OD) delta 7.75-7.68 (m, 1H), 6.58 (dd, 1H, J=4.0, 8.2 Hz), 3.47-3.36 (m, 2H), 3.27-3.20 (m, 1H), 3.11-3.05 (m, 1H), 3.01-2.96 (m, 1H, minor diastereomer), 2.69-2.62 (m, 1H), 2.57-2.51 (m 1H), 2.43-2.37 (m, 1H), 2.25-2.19 (m, 1H, minor diastereomer), 2.09-2.01 (m, 2H), 1.96-1.88 (m, 1H), 1.84-1.74 (m, 2H), 1.71-1.55 (m, 3H), 1.53-1.16 (m, 5H); ESI MS m/z 369 [C₂₁H₂₈N₄O₂+H]⁺; HPLC >99% (AUC), t_{R}=9.75 min.

### Example 49

### 2-(Bicyclo[2.2.1]heptan-2-yl)-7-hydroxy-N-(piperidin-3-yl)-1H-benzo[d]imidazole-4-carboxamide

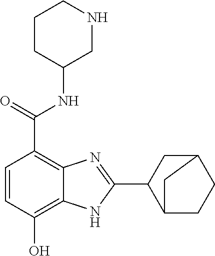

Following General Procedure C, tent-Butyl 3-(2-(bicyclo[2.2.1]heptan-2-yl)-7-methoxy-1H-benzo[d]imidazole-4-carboxamido)piperidine-1-carboxylate (210 mg crude) was reacted with boron tribromide to afford the desired product (72 mg, 43% yield) as a light yellow solid: ¹H NMR (300 MHz, CD₃OD) delta 7.69 (dd, J=3.6, 8.1 Hz, 1H), 6.61 (dd, J=2.7, 8.1 Hz, 1H), 4.12-4.01 (m, 1H), 3.45-3.36 (m, 1H), 3.03-2.93 (m, 1H), 2.78-2.52 (m, 3H), 2.44-2.36 (m, 1H), 2.25-1.16 (m 13H); ESI MS m/z 355 [C₂₀H₂₆N₄O₂ H]⁺; HPLC >99% (AUC), t_{R}=9.55 min (minor diastereomer), 9.74 min (major diastereomer).

### General Procedure D—Synthesis of Compounds of Formula (I-II) as Described in Scheme (1):

To a solution of acid (1.0 equiv) in DMF (5-10 mL) was added HATU (1.2-1.5 equiv), DIPEA (3.0-5.0 equiv), and the amine (1.5-2.0 equiv) and the reaction mixture was either stirred at room temperature for 16 h or heated at 50-70 degrees C. for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products. In some instances, the desired product was treated with TFA (1-2 mL) for 1 h, concentrated and purified by preparative HPLC

(C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products

### Example 50

### (S)-tert-Butyl 3-(2-(5-bromothiophen-2-yl)-7-hydroxy-1H-benzo[d]imidazole-4-carboxamido)piperidine-1-carboxylate

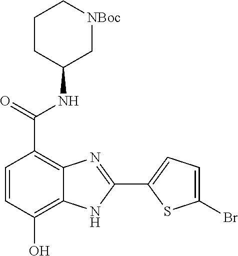

Following General Procedure D, 2-(5-bromothiophen-2-yl)-7-hydroxy-1H-benzo[d]imidazole-4-carboxylic acid (90 mg, 0.27 mmol) was reacted with (S)-tert-butyl 3-aminopiperidine-1-carboxylate (106 mg, 0.53 mmol) to afford the desired product (48 mg, 35% yield) as yellow-brown solid: ¹H NMR (500 MHz, CD₃OD) delta 7.84 (d, J=8.5 Hz, 1H), 7.71 (s, 1H), 7.28 (s, 1H), 6.78 (d, J=8.5 Hz, 1H), 4.21 (bs, 1H), 3.86 (bs, 1H), 3.58-3.18 m, 2H), 2.14-2.03 (m, 2H), 1.89 (bs, 1H), 1.59 (bs, 1H), 1.17 (bs, 1H); ESI MS m/z 521 [C₂₂H₂₅BrN₄O₄S]⁺; HPLC >99% (AUC), t_{R}=15.30 min.

### Example 51

### (S)-2-(5-bromothiophen-2-yl)-7-hydroxy-N-(piperidin-3-yl)-1H-benzo[d]imidazole-4-carboxamide

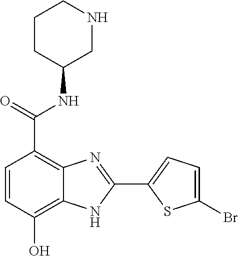

A solution of (S)-tert-butyl 3-(2-(5-bromothiophen-2-yl)-7-hydroxy-1H-benzo[d]imidazole-4-carboxamido)piperidine-1-carboxylate (35 mg, 0.067 mmol) in CH₂Cl₂ (1 mL) and TFA (1 mL) was stirred at room temperature for 1 h. The reaction mixture was concentrated and purified by purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired (20 mg, 72%) as yellow solid: ¹H NMR (500 MHz, DMSO-d₆) delta 13.61 (s, 1H), 11.00 (s, 1H), 9.57 (d, J=6.5 Hz, 1H), 8.75 (bs, 1H), 7.89 (d, J=4.0 Hz, 1H), 7.72 (d, J=8.0 Hz, 1H), 7.41 (d, J=3.5 Hz, 1H), 6.77 (d, J=8.5 Hz, 1H), 3.46 (d, J=8.5 Hz, 1H), 3.21 (d, J=12.5 Hz, 1H), 3.04-2.96 (m, 2H), 2.10 (bs, 1H), 2.03-2.00 (m, 2H), 1.85-1.70 (m, 4H), 0.68 (bs, 1H); ESI MS m/z 421 [C₁₇H₁₇BrN₄O₂S]⁺; HPLC 98.34% (AUC), t_{R}=8.17 min.

### Example 52

### 2-(Bicyclo[2.2.1]heptan-2-yl)-7-hydroxy-N-((S)-piperidin-3-yl)-1H-benzo[d]imidazole-4-carboxamide

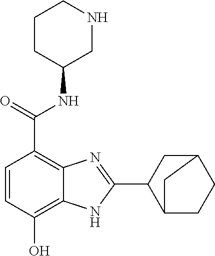

Following General Procedure C, (3S)-tert-Butyl 3-(2-(bicyclo[2.2.1]heptan-2-yl)-7-methoxy-1H-benzo[d]imidazole-4-carboxamido)piperidine-1-carboxylate (230 mg, crude) was reacted with boron tribromide to afford the desired product (103 mg, 52% over two steps) as a light brown solid: ¹H NMR (300 MHz, CD₃OD) delta 7.69 (dd, J=3.6, 8.4 Hz, 1H), 6.60 (dd, J=2.7, 8.4 Hz, 1H), 4.12-4.02 (m, 1H), 3.46-3.35 (m, 1H), 3.03-2.93 (m, 1H), 2.78-2.60 (m, 3H), 2.56-2.36 (m, 1H), 2.25-1.17 (m, 13H); ESI MS m/z 355 [C₂₀H₂₆N₄O₂+H]⁺; HPLC 99.0% (AUC), t_{R}=9.35 min (minor diastereomer), 9.49 min (major diastereomer).

### Example 53

### 2-(Bicyclo[2.2.1]heptan-2-yl)-7-hydroxy-N-(adamantane-3-ylamino)-1H-benzo[d]imidazole-4-carboxamide

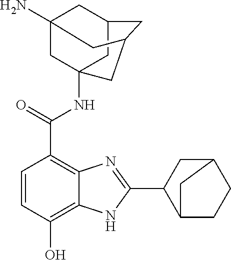

Following General Procedure C, tert-Butyl 3-{[2-(bicyclo[2.2.1]heptan-2-yl)-7-methoxy-1H-benzo[d]imidazole-4-carboxamido]methyl}adamantane-1-carboxylate (140 mg, crude) was reacted with boron tribromide to afford the desired product (57 mg, 31% over two steps) as a light yellow solid: ¹H NMR (300 MHz, CD₃OD) delta 7.66-7.62 (m, 1H), 6.57-6.53 (m, 1H), 3.45-3.35 (m, 1H), 3.00-2.90 (m, 1H, minor diastereomer), 2.68-2.62 (m, 1H, major diastereomer), 2.56-2.52 (m, 1H, minor diastereomer), 2.43-2.18 (m, 7H), 2.13-1.99 (m 3H), 1.84-1.21 (m, 12H); ESI MS m/z 421 [C₂₅H₃₂N₄O₂+H]⁺; HPLC 96.6% (AUC), t_{R}=10.45 min.

### Example 54

### 2-(Thiophene-2-yl)-7-hydroxy-N-(adamantate-3-ylamino)-1H-benzo[d]imidazole-4-carboxamide

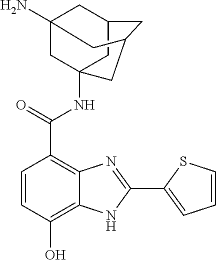

Following General Procedure C, tert-Butyl 3-((2-thiophene-2-yl)-7-methoxy-1H-benzo[d]imidazole-4-carboxamido)methyl)adamantane-1-carboxylate (110 mg) was reacted with boron tribromide to afford the desired product (62 mg, 28% over two steps) as a light yellow solid: ¹H NMR (300 MHz, CD₃OD) delta 7.80 (d, J=3.9 Hz, 1H), 7.69 (d, J=8.4 Hz, 1H), 7.58 (d, 4.8 Hz), 7.20-7.17 (m, 1H), 6.59 (d, 1H, J=8.4 Hz), 2.38-2.11 (m, 8H), 1.86-1.63 (m, 6H); ESI MS m/z 409 [C₂₂H₂₄N₄O₂S+H]⁺; HPLC >99% (AUC), t_{R}=11.27 min.

### Example 55

### N-(3-Aminocyclohexyl)-2-(bicyclo[2.2.1]heptan-2-yl)-7-hydroxy-1H-benzo[d]imidazole-4-carboxamide

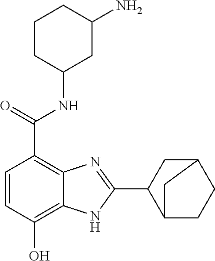

Following General Procedure C, tert-Butyl 3-(2-(bicyclo[2.2.1]heptan-2-yl)-7-methoxy-1H-benzo[d]imidazole-4-carboxamido)cyclohexylcarbamate (120 mg, crude) was reacted with boron tribromide to afford the desired product (66 mg, 40% yield) as a light yellow solid: ¹H NMR (300 MHz, CD₃OD) delta 7.72-7.67 (m, 1H), 6.58-6.55 (m, 1H), 4.57-4.48 (m, 1H, minor diastereomer), 4.03-3.90 (m, 1H, major diastereomer), 3.45-3.35 (m, 1H), 3.03-2.90 (m, 1H), 2.66-2.52 (m, 1H), 2.44-2.32 (m 2H, major diastereomer), 2.22-1.14 (m, 15H); ESI MS m/z 369 [C₂₁H₂₈N₄O₂+H]⁺; HPLC >99% (AUC), t_{R}=9.40, 9.53, 9.58, 9.81 min (mixture of diastereomers).

### Example 56

### N-{[(cis)-4-Aminocyclohexyl]methyl}-2-(bicyclo[2.2.1]heptan-2-yl)-7-hydroxy-1H-benzo[d]imidazole-4-carboxamide

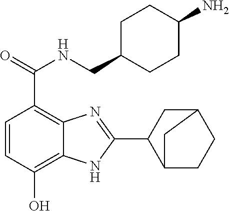

Following General Procedure C, tert-Butyl (cis)-4-{[2-(bicyclo[2.2.1]heptan-2-yl)-7-methoxy-1H-benzo[d]imidazole-4-carboxamido]methy 1}cyclohexylcarbamate (220 mg crude) was reacted with boron tribromide to afford the desired product (64 mg, 53% over two steps) as a light yellow solid: ¹H NMR (300 MHz, CD₃OD) delta 7.69 (dd, J=3.9, 8.4 Hz, 1H), 6.59-6.54 (m, 1H), 3.56-3.37 (m, 2H), 3.15-3.07 (m, 1H), 3.00-2.90 (m, 1H, minor diastereomer), 2.74-2.66 (m, 1H, minor diastereomer), 2.55-2.51 (m, 1H, minor diastereomer), 2.42-2.34 (m, 1H), 2.25-2.16 (m, 1H, minor diastereomer), 2.06-1.98 (m, 1H), 1.80-1.20 (m, 14H); ESI MS m/z 383 [C₂₂H₃₀N₄O₂+H]⁺; HPLC 99.0% (AUC), t_{R}=9.53, 9.88, 9.96 min (mixture of diastereomers).

### Example 57

### (S)-7-hydroxy-2-(5-(piperazin-1-yl)thiophen-2-yl)-N-(piperidin-3-yl)-1H-benzo[d]imidazole-4-carboxamide

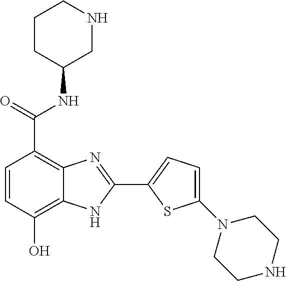

A mixture of (S)-tert-Butyl 3-(2-(5-bromothiophen-2-yl)-7-hydroxy-1H-benzo[d]imidazole-4-carboxamido)piperidine-1-carboxylate (0.12 g, 0.24 mmol), tert-butyl piperazine-1-carboxylate (110 mg, 0.60 mmol), CuI (5.7 mg, 0.030 mmol), Cu (2.0 mg, 0.030 mmol), K₃PO₄.H₂O (160 mg, 0.72 mmol) in 2-(dimethylamino)ethanol (2 mL) was stirred at 75 degrees C. for 18 h. The reaction mixture was cooled, concentrated, dissolved in CH₃OH (3 mL) and filtered. The filtrate was purified by preparative HPLC (C18 silica, 10-90% acetonitrile/water with 0.05% TFA). The desired fractions were combined, concentrated and the residue was dissolved in CH₂Cl₂ (2 mL) and TFA (1 mL) and stirred at rt for 30 min. The reaction mixture was concentrated and the residue was eluted through an ion-exchange column (SCX-2) (using methanol and 7 N methanol in ammonia) to obtain the desired product (7 mg, 14% yield) as a yellow solid: ¹H NMR (500 MHz, CD₃OD) delta 8.20 (d, J=4.5 Hz, 1H), 7.50 (d, J=4.5 Hz, 1H), 7.14 (d, J=4.0 Hz, 1H), 6.54 (d, J=3.5 Hz, 1H), 4.20-4.16 (m, 1H), 3.43 (dd, J=12.5, 3.5 Hz, 1H), 3.19-3.15 (m, 2H), 3.10-2.97 (m, 3H), 2.05-1.96 (m, 2H), 1.84-1.72 (m, 3H), 1.19-1.16 (m, 1H), 1.13-1.08 (m, 1H); ESI MS m/z 427 [C₂₁H₂₆N₆O₂S+H]⁺ HPLC 97.13% (AUC), t_{R}=8.29 min.

### Example 58

### (R)-7-hydroxy-N-(piperidin-3-ylmethyl)-2-(thiophen-2-ylmethyl)-1H-benzo[d]imidazole-4-carboxamide

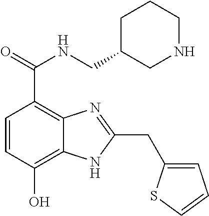

Following General Procedure D, 2-(5-bromothiophen-2-yl)-7-hydroxy-1H-benzo[d]imidazole-4-carboxylic acid (0.13 mg, 0.47 mmol) was reacted with (S)-tert-butyl 3-(aminomethyl)piperidine-1-carboxylate (200 mg, 0.93 mmol) and the intermediate was treated with TFA to afford the desired product (15 mg, 31% yield) as yellow solid: ¹H NMR (500 MHz, CD₃OD) delta 7.75 (d, J=8.5 Hz, 1H), 7.30 (dd, J=5.5, 1.5 Hz, 1H), 7.02-7.01 (m, 1H), 6.98 (dd, J=5.0, 3.5 Hz, 1H), 6.70 (d, J=8.5 Hz, 1H), 4.52 (s, 2H), 3.53-3.45 (m, 2H), 3.37 (dd, J=9.0, 6.0 Hz, 1H), 2.95-2.89 (m, 2H), 2.82 (t, J=12.0 Hz, 1H), 2.15-2.11 (m, 1H), 2.00-1.94 (m, 3H), 1.78-1.74 (m, 1H), 1.44-1.36 (m, 2H); ESI MS m/z 371 [C₁₉H₂₂N₄O₂S+H]⁺ HPLC 95.5% (AUC), t_{R}=7.17 min.

### Example 59

### (S)-7-hydroxy-N-(piperidin-3-yl)-2-(thiophen-2-ylmethyl)-1H-benzo[d]imidazole-4-carboxamide

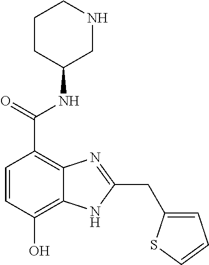

Following General Procedure D, 2-(5-bromothiophen-2-yl)-7-hydroxy-1H-benzo[d]imidazole-4-carboxylic acid (0.17 mg, 0.62 mmol) was reacted with (S)-tert-butyl 3-aminopiperidine-1-carboxylate (250 mg, 1.3 mmol) and the intermediate was treated with TFA to afford the desired product (25 mg, 68% yield) as yellow solid: ¹H NMR (500 MHz, CD₃OD) delta 7.63 (d, J=8.5 Hz, 1H), 7.20 (dd, J=5.0, 1.0 Hz, 1H), 6.91 (dd, J=5.0, 3.5 Hz, 1H), 6.57 (d, J=8.5 Hz, 1H), 4.37 (s, 2H), 4.15-4.11 (m, 1H), 3.37 (dd, J=10.5, 3.5 Hz, 1H), 3.14-3.11 (m, 1H), 2.93-2.86 (m, 2H), 2.04-2.01 (m, 1H), 1.94-1.91 (m, 1H), 1.74-1.65 (m, 2H). ESI MS m/z 357 [C₁₈H₂₀N₄O₂S+H]⁺ HPLC 96.59% (AUC), t_{R}=7.07 min.

### Example 60

### (S)-7-hydroxy-N-(piperidin-3-ylmethyl)-2-(thiophen-2-ylmethyl)-1H-benzo[d]imidazole-4-carboxamide

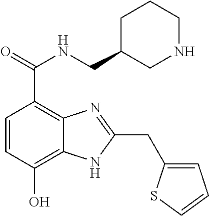

Following General Procedure D, 2-(5-bromothiophen-2-yl)-7-hydroxy-1H-benzo[d]imidazole-4-carboxylic acid (0.13 mg, 0.47 mmol) was reacted with (R)-tert-butyl 3-(aminomethyl)piperidine-1-carboxylate (200 mg, 0.93 mmol) and the intermediate was treated with TFA to afford the desired product (12 mg, 28% yield) as yellow solid: ¹H NMR (500 MHz, CD₃OD) delta 7.75 (d, J=8.5 Hz, 1H), 7.30 (dd, J=5.5, 1.5 Hz, 1H), 7.02-7.01 (m, 1H), 6.98 (dd, J=5.0, 3.5 Hz, 1H), 6.70 (d, J=5.0 Hz, 1H), 4.50 (s, 2H), 3.51-3.48 (m, 2H), 3.37 (dd, J=13.0, 7.0 Hz, 1H), 2.92-2.89 (m, 2H), 2.80 (t, J=12.0 Hz, 1H), 2.16-2.10 (m, 1H), 2.00-1.95 (m, 3H), 1.80-1.72 (m, 1H), 1.44-1.39 (m, 2H); ESI MS m/z 371 [C₁₉H₂₂N₄O₂S+H]⁺ HPLC 96.8% (AUC), t_{R}=6.93 min.

### Example 61

### Step 1: Synthesis of Methyl 3-(5-bromothiophene-2-carboximidamido)-4-methoxybenzoate Hydrochloride

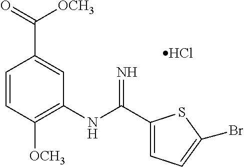

Following the procedure outlined for step 1 in Example 1, methyl 3-amino-4-methoxybenzoate (1.5 g, 7.9 mmol) was reacted with 5-bromothiophene-2-carbonitrile (3.0 g, 16 mmol) to afford the desired product (1.6 g, 54% yield) as a dark brown solid: ESI MS m/z 368 [C₁₄H₁₃BrN₂O₃S+H]⁺.

### Step 2: Synthesis of Methyl 2-(5-bromothiophen-2-yl)-7-methoxy-1H-benzo[d]imidazole-4-carboxylate

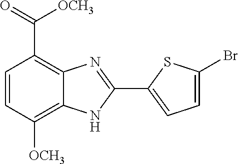

Following the procedure outlined for step 2 in Example 1, methyl 3-(5-bromothiophene-2-carboximidamido)-4-methoxybenzoate hydrochloride (1.7 g, 4.2 mmol) was reacted with 5% aq NaOCl and satd. aq NaHCO₃ to afford the desired product (0.45 g, 30% yield) as a brown solid: ESI MS m/z 369 [C₁₄H₁₁BrN₂O₃S+H]⁺.

### Step 3: Synthesis of 2-(5-Bromothiophen-2-yl)-7-hydroxy-1H-benzo[d]imidazole-4-carboxylic Acid

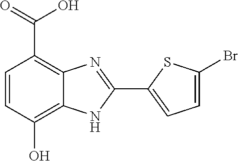

Following the procedure outlined for step 4 in Example 1, methyl 2-(5-bromothiophen-2-yl)-7-methoxy-1H-benzo[d]imidazole-4-carboxylate (0.40 g, 1.1 mmol) was reacted with boron tribromide (1.5 g, 6.6 mmol) to afford the desired product (0.34 g, 92% yield) as a light brown solid: ESI MS m/z 340 [C₁₂H₇BrN₂O₃S+H]⁺.

### Example 62

### Step 1: Synthesis of Methyl 4-methoxy-3-(2-(thiophen-2-yl)acetimidamido)benzoate Hydrochloride

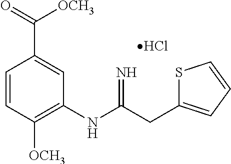

Following the procedure outlined for step 1 in Example 1, methyl 3-amino-4-methoxybenzoate (2.2 g, 12 mmol) was reacted with 2-(thiophen-2-yl)acetonitrile (3.0 g, 24 mmol) to afford the desired product (3.2 g, 78% yield) as a yellow brown solid: ESI MS m/z 305 [C₁₅H₁₆N₂O₃S+H]⁺.

### Step 2: Synthesis of Methyl 7-methoxy-2-(thiophen-2-ylmethyl)-1H-benzo[d]imidazole-4-carboxylate

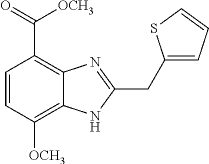

Following the procedure outlined for step 2 in Example 1, methyl 4-methoxy-3-(2-(thiophen-2-yl)acetimidamido)benzoate hydrochloride (3.1 g, 10 mmol) was reacted with 5% aq NaOCl and satd. aq NaHCO₃ to afford the desired product (1.1 g, 30% yield) as a brown solid: ESI MS m/z 303 [C₁₅H₁₄N₂O₃S+H]⁺.

### Step 3: Synthesis of 7-hydroxy-2-(thiophen-2-ylmethyl)-1H-benzo[d]imidazole-4-carboxylic Acid

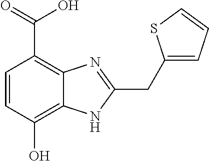

Following the procedure outlined for step 4 in Example 1, methyl 7-methoxy-2-(thiophene-2-ylmethyl)-1H-benzo[d]imidazole-4-carboxylate (0.91 g, 3.0 mmol) was reacted with boron tribromide (4.5 g, 18 mmol) to afford the desired product (0.63 g, 73% yield) as a light brown solid: ESI MS m/z 275 [C₁₃H₁₀N₂O₃S+H]⁺.

### Example 63

### Step 1: Synthesis of Methyl 3-(bicyclo[2.2.1]heptane-2-carboximidamido)-4-methoxybenzoate

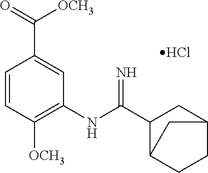

Following the procedure outlined for step 1 in Example 1, methyl-3-amino-4-methoxy benzoate (7.5 g, 41 mmol) was reacted with 2-norbornane carbonitrile (10 g, 82 mmol) to afford product (11 g, 90%) as a white solid: ¹H NMR (300 MHz, DMSO-d₆) delta 8.29-8.20 (m, 1H), 7.99-7.96 (m, 1H), 7.33-7.28 (m, 1H), 3.88 (s, 3H), 3.84 (s, 3H), 2.70-2.62 (m, 1H), 1.87-1.17 (m, 8H); ESI MS m/z 303 [C₁₇H₂₂N₂O₃+H]⁺.

### Step 2: Synthesis of Methyl 2-(bicyclo[2.2.1]heptan-2-yl)-7-methoxy-1H-benzo[d]imidazole-4-carboxylate

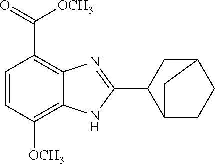

Following the procedure outlined for step 2 in Example 1, methyl 3-(bicyclo[2.2.1]heptane-2-carboximidamido)-4-methoxybenzoate (11 g, 37 mmol) was reacted with NaOCl (33 mL, 10-13%, 44 mmol) and chromatographed (hexanes/ethyl acetate) to afford product (3.9 g, 36%) as a foam: ¹H NMR (300 MHz, DMSO-d₆) delta 12.05 (s, 1H, tautomer 1), 11.97 (s, 1H, tautomer 2), 7.73 (dd, 1H, J=1.2, 8.7 Hz), 6.78 (dd, 1H, J=2.4, 8.7 Hz), 4.00 (s, 3H, tautomer 1), 3.98 (s, 3H, tautomer 2), 3.90 (s, 3H, tautomer 1), 3.89 (s, 3H, tautomer 2), 3.47-3.41 (m, 1H, tautomer 1), 3.11-3.06 (m, 11-1, tautomer 2), 2.70-2.66 (m, 11-1, tautomer 1), 2.38-2.18 (m, 2H), 2.08-2.00 (m, 1H, tautomer 1), 1.91-1.80 (m, 1H, tautomer 2), 1.68-1.24 (m, 5H), 1.11-0.98 (m, 1H); ESI MS m/z 301 [C₁₇H₂₀N₂O₃+H]⁺.

### Step 3: Synthesis of 2-(Bicyclo[2.2.1]heptan-2-yl)-7-methoxy-1H-benzo[d]imidazole-4-carboxylic Acid

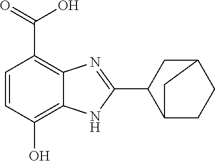

Following the procedure outlined for step 3 in Example 1, methyl 2-(bicyclo[2.2.1]heptan-2-yl)-7-methoxy-1H-benzo[d]imidazole-4-carboxylate (3.9 g, 13 mmol) was reacted with sodium hydroxide (30 mL, 3 M) to afford crude product (3.6 g) as a white solid: ESI MS m/z 287 [C₁₆H₁₈N₂O₃+H]⁺.

### Example 64

### tert-Butyl 3-{[2-(bicyclo[2.2.1]heptan-2-yl)-7-methoxy-1H-benzo[d]imidazole-4-carboxamido]methyl}piperidine-1-carboxylate

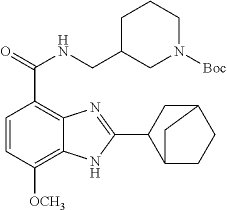

Following General Procedure D 2-(bicyclo[2.2.1]heptan-2-yl)-7-methoxy-1H-benzo[d]imidazole-4-carboxylic acid (125 mg, 0.43 mmol) was reacted with tert-butyl 3-(aminomethyl)piperidine-1-carboxylate (138 mg, 0.65 mmol) to afford the desire product (338 mg, crude) as an oil: ESI MS m/z 483 [C₂₇H₃₈N₄O₄+H]⁺.

### Example 65

### tert-Butyl 3-((2-(bicyclo[2.2.1]heptan-2-yl)-7-methoxy-1H-benzo[d]imidazole-4-carboxamido)methyl)adamantane-1-carboxylate

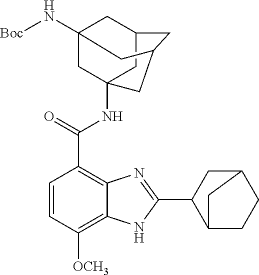

Following General Procedure D 2-(bicyclo[2.2.1]heptan-2-yl)-7-methoxy-1H-benzo[d]imidazole-4-carboxylic acid (125 mg, 0.43 mmol) was reacted with tert-butyl 3-aminoadamantanecarboxylate (176 mg, 0.65 mmol) to afford the desire product (145 mg crude) as an oil: ESI MS m/z 535 [C₃₁H₄₂N₄O₄+H]⁺.

### Example 66

### (3S)-tert-Butyl 3-(2-(bicyclo[2.2.1]heptan-2-yl)-7-methoxy-1H-benzo[d]imidazole-4-carboxamido)piperidine-1-carboxylate

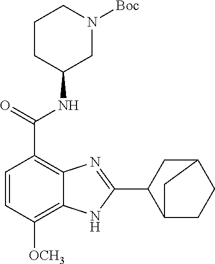

Following General Procedure D 2-(bicyclo[2.2.1]heptan-2-yl)-7-methoxy-1H-benzo[d]imidazole-4-carboxylic acid (150 mg, 0.54 mmol) was reacted with (S)-tert-butyl 3-aminopiperidine-1-carboxylate (160 mg, 0.81 mmol) to afford the desire product (237 mg crude) as an oil: ESI MS m/z 467 [C₂₆H₃₆N₄O₄+H]⁺.

### Example 67

### tert-Butyl (cis)-4-((2-(bicyclo[2.2.1]heptan-2-yl)-7-methoxy-1H-benzo[d]imidazole-4-carboxamido)methyl)cyclohexylcarbamate

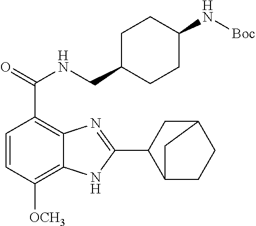

Following General Procedure D 2-(bicyclo[2.2.1]heptan-2-yl)-7-methoxy-1H-benzo[d]imidazole-4-carboxylic acid (90 mg, 0.31 mmol) was reacted with tert-butyl (1s,4s)-4-(aminomethyl)cyclohexylcarbamate (71 mg, 0.31 mmol) to afford the desire product (237 mg crude) as an oil: ESI MS m/z 497 [C₂₈H₄₀N₄O₄+H]⁺.

### Example 68

### tert-Butyl 3-((2-thiophene-2-yl)-7-methoxy-1H-benzo[d]imidazole-4-carboxamido)methyl)adamantane-1-carboxylate

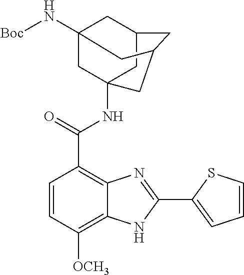

Following General Procedure B, 7-methoxy-2-(thiophen-2-yl)-1H-benzo[d]imidazole-4-carboxylic acid (0.15 g, 0.55 mmol) was reacted with tert-butyl 3-aminoadamantanecarboxylate (0.22 g, 0.82 mmol) to afford the desired product (118 mg crude) as a white solid: ESI MS m/z 523 [C₂₈H₃₄N₄O₄S+H]⁺.

### Example 69

### tert-Butyl 3-(2-(bicyclo[2.2.1]heptan-2-yl)-7-methoxy-1H-benzo[d]imidazole-4-carboxamido)piperidine-1-carboxylate

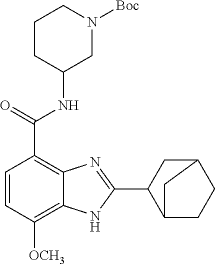

Following General Procedure B, 2-(Bicyclo[2.2.1]heptan-2-yl)-7-methoxy-1H-benzo[d]imidazole-4-carboxylic acid (150 mg, 0.55 mmol) was reacted with tert-butyl 3-aminopiperidine-1-carboxylate (0.22 g, 1.1 mmol) to afford the desired product (219 mg crude) as a foam: ESI MS m/z 469 [C₂₆H₃₆N₄O₄+H]⁺.

### Example 70

### tert-Butyl 3-(2-(bicyclo[2.2.1]heptan-2-yl)-7-methoxy-1H-benzo[d]imidazole-4-carboxamido)cyclohexylcarbamate

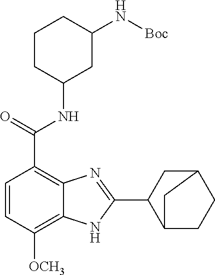

Following General Procedure B, 2-(Bicyclo[2.2.1]heptan-2-yl)-7-methoxy-1H-benzo[d]imidazole-4-carboxylic acid (150 mg, 0.55 mmol) was reacted with tert-butyl 3-aminocyclohexylcarbamate (0.24 g, 1.1 mmol) to afford the desired product (126 mg crude) as a glass: ESI MS m/z 483 [C₂₇H₃₈N₄O₄+H]⁺.

### Examples 71

### Kinase Assay

PBK activity was determined in the presence or absence of compounds using fluorescein isothiocyanate-labeled (FITC-labeled) histone H3 peptide as a substrate. The extent of FITC-labeled histone 113 peptide phosphorylation was measured by immobilized metal ion affinity-based fluorescence polarization (IMAP) technology (Sportsman J R, et al., Assay Drug Dev. Technol. 2: 205-14, 2004) using IMAP FP Progressive Binding System (Molecular Devices Corporation). Test compounds were dissolved in DMSO at 12.5 mM and then serially diluted as the DMSO concentration in the assays to be 1%. The serially diluted compounds, 0.8 ng/micro-L PBK (Carna Biosciences) and 100 nM FITC-labeled histone H3 peptide were reacted in a reaction buffer (20 mM HEPES, 0.01% Tween-20, 0.3 mM MgCl₂, 2 mM dithiothreitol, 50 micro-M ATP, pH 7.4) at room temperature for 1 hour. The reaction was stopped by the addition of three fold assay volume of progressive binding solution. Following 0.5 hour incubation at room temperature, fluorescence polarization was measured by Wallac EnVision 2103 multilabel reader (PerkinElmer). IC50 values were calculated by nonlinear four parameter fit using SigmaPlot, version 10.0 (Systat Software, Inc.). IC₅₀ values of the typical compounds of the present invention are shown in following table 2:

TABLE 2

| | | IC50 (microM) |
|---|---|---|
| Example No. | Compound | (kinase assay) |
| 38 | (S)-7-Hydroxy-N-(piperidin-3-yl)-2-(thiophen-2-yl)-1H- | 0.086 |
| | benzo[d]imidazole-4-carboxamide | |
| 35 | 7-Hydroxy-N-(piperidin-3-yl)-2-(thiophen-2-yl)-1H- | 0.18 |
| | benzo[d]imidazole-4-carboxamide | |
| 39 | 7-Hydroxy-N-(piperidin-3-ylmethyl)-2-(thiophen-2-yl)-1H- | 0.2 |
| | benzo[d]imidazole-4-carboxamide | |
| 9 | (S)-2-cyclopropyl-4-hydroxy-N-(piperidin-3-yl)-1H- | 0.2 |
| | benzo[d]imidazole-7-carboxamide | |
| 16 | (S)-2-Cyclopentyl-4-hydroxy-N-(piperidin-3-yl)-1H- | 0.27 |
| | benzo[d]imidazole-7-carboxamide | |
| 17 | (S)-4-Hydroxy-2-phenyl-N-(piperidin-3-yl)-1H- | 0.3 |
| | benzo[d]imidazole-7-carboxamide | |
| 11 | 2-cyclopropyl-4-hydroxy-N-(piperidin-3-yl)-1H- | 0.41 |
| | benzo[d]imidazole-7-carboxamide | |
| 47 | N-(4-Aminocyclohexyl)-7-hydroxy-2-(thiophen-2-yl)-1H- | 0.52 |
| | benzo[d]imidazole-4-carboxamide | |
| 15 | 2-Cyclopentyl-4-hydroxy-N-(piperidin-3-ylmethyl)-1H- | 0.59 |
| | benzo[d]imidazole-7-carboxamide | |
| 45 | 7-Hydroxy-N-(piperidin-2-ylmethyl)-2-(thiophen-2-yl)-1H- | 0.62 |
| | benzo[d]imidazole-4-carboxamide | |
| 7 | 2-Cyclopropyl-4-hydroxy-N-(piperidin-2-ylmethyl)-1H- | 0.62 |
| | benzo[d]imidazole-7-carboxamide | |
| 46 | 7-Hydroxy-N-(pyrrolidin-3-ylmethyl)-2-(thiophen-2-yl)-1H- | 0.63 |
| | benzo[d]imidazole-4-carboxamide | |
| 44 | 7-Hydroxy-N-(pyrrolidin-3-yl)-2-(thiophen-2-yl)-1H- | 0.73 |
| | benzo[d]imidazole-4-carboxamide | |
| 19 | 4-hydroxy-2-phenyl-N-(piperidin-3-ylmethyl)-1H- | 0.91 |
| | benzo[d]imidazole-7-carboxamide | |
| 43 | N-(Azetidin-3-ylmethyl)-7-hydroxy-2-(thiophen-2-yl)-1H- | 0.97 |
| | benzo[d]imidazole-4-carboxamide | |
| 12 | 2-cyclopropyl-4-hydroxy-N-(pyrrolidin-3-yl)-1H- | 1.3 |
| | benzo[d]imidazole-7-carboxamide | |
| 13 | N-(azetidin-3-ylmethyl)-2-cyclopropyl-4-hydroxy-1H- | 1.6 |
| | benzo[d]imidazole-7-carboxamide | |
| 42 | 7-Hydroxy-N-(piperidin-4-ylmethyl)-2-(thiophen-2-yl)-1H- | 1.7 |
| | benzo[d]imidazole-4-carboxamide | |
| 40 | 7-Hydroxy-N-(piperidin-4-yl)-2-(thiophen-2-yl)-1H- | 1.8 |
| | benzo[d]imidazole-4-carboxamide | |
| 10 | 2-cyclopropyl-4-hydroxy-N-(piperidin-4-yl)-1H- | 2.6 |
| | benzo[d]imidazole-7-carboxamide | |
| 5 | 2-Cyclopropyl-4-hydroxy-N-(piperidin-4-ylmethyl)-1H- | 2.9 |
| | benzo[d]imidazole-7-carboxamide | |
| 18 | 4-Hydroxy-2-phenyl-N-(piperidin-2-ylmethyl)-1H- | 2.9 |
| | benzo[d]imidazole-7-carboxamide | |
| 36 | 7-Hydroxy-N-[2-(piperazin-1-yl)ethyl]-2-(thiophen-2-yl)-1H- | 3 |
| | benzo[d]imidazole-4-carboxamide | |
| 37 | (R)-7-Hydroxy-N-(piperidin-3-yl)-2-(thiophen-2-yl)-1H- | 3.2 |
| | benzo[d]imidazole-4-carboxamide | |
| 41 | 7-Hydroxy-N-(1-methylpiperidin-3-yl)-2-(thiophen-2-yl)-1H- | 4.5 |
| | benzo[d]imidazole-4-carboxamide | |
| 6 | 2-Cyclopropyl-4-hydroxy-N-(piperidin-3-yl-methyl)-1H- | 6.2 |
| | benzo[d]imidazole-7-carboxamide | |
| 14 | 2-cyclopentyl-4-hydroxy-N-(piperidin-2-ylmethyl)-1H- | 10 |
| | benzo[d]imidazole-7-carboxamide | |
| 20 | 7-Hydroxy-N-(4-hydroxycyclohexyl)-2-(thiophen-2-yl)-1H- | 17 |
| | benzo[d]imidazole-4-carboxamide | |
| 21 | (7-hydroxy-2-[thiophen-2-yl]-1H-benzo[d]imidazol-4- | 18 |
| | yl)(piperazin-1-yl)methanone | |
| 8 | 2-cyclopropyl-4-hydroxy-N-(1-methylpiperidin-3-yl)-1H- | 26 |
| | benzo[d]imidazole-7-carboxamide | |
| 48 | 2-(Bicyclo[2.2.1]heptan-2-yl)-7-hydroxy-N-(piperidin-3-ylmethyl)- | 1.1 |
| | 1H-benzo[d]imidazole-4-carboxamide | |
| 49 | 2-(Bicyclo[2.2.1]heptan-2-yl)-7-hydroxy-N-(piperidin-3-yl)-1H- | 0.85 |
| | benzo[d]imidazole-4-carboxamide | |
| 50 | (S)-tert-Butyl | 100 |
| | 3-(2-(5-bromothiophen-2-yl)-7-hydroxy-1H-benzo[d]imidazole- | |
| | 4-carboxamido)piperidine-1-carboxylate | |
| 51 | (S)-2-(5-bromothiophen-2-yl)-7-hydroxy-N-(piperidin-3-yl)-1H- | 0.77 |
| | benzo[d]imidazole-4-carboxamide | |
| 52 | 2-(Bicyclo[2.2.1]heptan-2-yl)-7-hydroxy-N-((S)-piperidin-3-yl)- | 0.45 |
| | 1H-benzo[d]imidazole-4-carboxamide | |
| 53 | 2-(Bicyclo[2.2.1]heptan-2-yl)-7-hydroxy-N-(adamantane-3- | 0.5 |
| | ylamino)-1H-benzo[d]imidazole-4-carboxamide | |
| 54 | 2-(Thiophene-2-yl)-7-hydroxy-N-(adamantate-3-ylamino)-1H- | 0.19 |
| | benzo[d]imidazole-4-carboxamide | |
| 55 | N-(3-Aminocyclohexyl)-2-(bicyclo[2.2.1]heptan-2-yl)-7-hydroxy- | 0.57 |
| | 1H-benzo[d]imidazole-4-carboxamide | |
| 56 | N-{[(cis)-4-Aminocyclohexyl]methyl}-2-(bicyclo[2.2.1]heptan- | 2.2 |
| | 2-yl)-7-hydroxy-1H-benzo[d]imidazole-4-carboxamide | |
| 57 | (S)-7-hydroxy-2-(5-(piperazin-1-yl)thiophen-2-yl)-N-(piperidin- | 3.2 |
| | 3-yl)-1H-benzo[d]imidazole-4-carboxamide | |
| 58 | (R)-7-hydroxy-N-(piperidin-3-ylmethyl)-2-(thiophen-2-ylmethyl)- | 0.69 |
| | 1H-benzo[d]imidazole-4-carboxamide | |
| 59 | (S)-7-hydroxy-N-(piperidin-3-yl)-2-(thiophen-2-ylmethyl)-1H- | 0.5 |
| | benzo[d]imidazole-4-carboxamide | |
| 60 | (S)-7-hydroxy-N-(piperidin-3-ylmethyl)-2-(thiophen-2-ylmethyl)- | 0.55 |
| | 1H-benzo[d]imidazole-4-carboxamide | |

### Examples 72

### Western Blot Analysis

To evaluate the expression status of PBK in several cell lines, western blot analysis was performed using crude cell lysate collected from those cells. Anti-PBK antibody (clone 31, BD Biosciences) was used to visualize the expression. Breast cancer cell lines, T47D and BT-549 expressed PBK significantly although Bladder cancer cell line and HT-1197 showed no expression of PBK.

### Examples 73

### Cell-Based Assay

Active candidate inhibitors against PBK were evaluated for their target-specific cytotoxicity using T47D, BT-549, and HT-1197 cells was used for negative control. 100 micro-L of cell suspension was seeded onto 96-well microtiter plate (ViewPlate-96FTC, PerkinElmer). The initial cell concentration of T47D, BT-549 and HT-1197 were 3,000 cells/well, 2,000 cells/well and 2,500 cells/well, respectively. Cellular growth was determined using Cell Counting Kit-8 (DOJINDO) at 72 hours after the exposure of the candidate inhibitors. IC50 was used as an indicator of the anti-proliferative activity of the inhibitors, and calculated by serial dilution method (0, 1.5625, 3.125, 6.25, 12.5, 25, 50, and 100 micro-M). Accurate IC50 values were calculated as described previously.

IC₅₀ values of the typical compounds of the present invention are shown in following table 3:

TABLE 3

| | | IC50 | IC50 | IC50 |
|---|---|---|---|---|
| | | (microM) | (microM) | (microM) |
| Example No. | Compound | (BT549) | (T47D) | (HT1197) |
| 16 | (S)-2-Cyclopentyl-4-hydroxy-N-(piperidin-3-yl)- | 0.37 | 2.6 | 19 |
| | 1H-benzo[d]imidazole-7-carboxamide | | | |
| 38 | (S)-7-Hydroxy-N-(piperidin-3-yl)-2-(thiophen-2- | 0.46 | 0.36 | 33 |
| | yl)-1H-benzo[d]imidazole-4-carboxamide | | | |
| 9 | (S)-2-cyclopropyl-4-hydroxy-N-(piperidin-3-yl)- | 0.73 | 1.5 | 7.1 |
| | 1H-benzo[d]imidazole-7-carboxamide | | | |
| 35 | 7-Hydroxy-N-(piperidin-3-yl)-2-(thiophen-2-yl)- | 0.77 | 0.81 | 49 |
| | 1H-benzo[d]imidazole-4-carboxamide | | | |
| 11 | 2-cyclopropyl-4-hydroxy-N-(piperidin-3-yl)-1H- | 1.6 | 3.2 | 44 |
| | benzo[d]imidazole-7-carboxamide | | | |
| 47 | N-(4-Aminocyclohexyl)-7-hydroxy-2-(thiophen-2- | 5.3 | 6.2 | 58 |
| | yl)-1H-benzo[d]imidazole-4-carboxamide | | | |
| 41 | 7-Hydroxy-N-(1-methylpiperidin-3-yl)-2-(thiophen- | 8.1 | 9.8 | 24 |
| | 2-yl)-1H-benzo[d]imidazole-4-carboxamide | | | |
| 7 | 2-Cyclopropyl-4-hydroxy-N-(piperidin-2-ylmethyl)- | 9.5 | 11 | >100 |
| | 1H-benzo[d]imidazole-7-carboxamide | | | |
| 39 | 7-Hydroxy-N-(piperidin-3-ylmethyl)-2-(thiophen- | 15 | 8.6 | >100 |
| | 2-yl)-1H-benzo[d]imidazole-4-carboxamide | | | |
| 44 | 7-Hydroxy-N-(pyrrolidin-3-yl)-2-(thiophen-2-yl)- | 15 | 20 | >100 |
| | 1H-benzo[d]imidazole-4-carboxamide | | | |
| 37 | (R)-7-Hydroxy-N-(piperidin-3-yl)-2-(thiophen-2- | 20 | 11 | >100 |
| | yl)-1H-benzo[d]imidazole-4-carboxamide | | | |
| 10 | 2-cyclopropyl-4-hydroxy-N-(piperidin-4-yl)-1H- | 20 | 19 | >100 |
| | benzo[d]imidazole-7-carboxamide | | | |
| 15 | 2-Cyclopentyl-4-hydroxy-N-(piperidin-3-ylmethyl)- | 25 | 21 | >100 |
| | 1H-benzo[d]imidazole-7-carboxamide | | | |
| 45 | 7-Hydroxy-N-(piperidin-2-ylmethyl)-2-(thiophen- | 29 | 7.8 | >100 |
| | 2-yl)-1H-benzo[d]imidazole-4-carboxamide | | | |
| 6 | 2-Cyclopropyl-4-hydroxy-N-(piperidin-3-yl-methyl)- | 30 | 55 | >100 |
| | 1H-benzo[d]imidazole-7-carboxamide | | | |
| 18 | 4-Hydroxy-2-phenyl-N-(piperidin-2-ylmethyl)-1H- | 31 | 15 | 98 |
| | benzo[d]imidazole-7-carboxamide | | | |
| 12 | 2-cyclopropyl-4-hydroxy-N-(pyrrolidin-3-yl)-1H- | 47 | 58 | >100 |
| | benzo[d]imidazole-7-carboxamide | | | |
| 40 | 7-Hydroxy-N-(piperidin-4-yl)-2-(thiophen-2-yl)- | 73 | 13 | >100 |
| | 1H-benzo[d]imidazole-4-carboxamide | | | |
| 48 | 2-(Bicyclo[2.2.1]heptan-2-yl)-7-hydroxy-N- | 20 | 49 | 100 |
| | (piperidin-3-ylmethyl)-1H-benzo[d]imidazole-4- | | | |
| | carboxamide | | | |
| 49 | 2-(Bicyclo[2.2.1]heptan-2-yl)-7-hydroxy-N-piperidin- | 0.65 | 4.1 | 14 |
| | (3-yl)-1H-benzo[d]imidazole-4-carboxamide | | | |
| 51 | (S)-2-(5-bromothiophen-2-yl)-7-hydroxy-N- | 0.18 | 0.14 | 5.7 |
| | (piperidin-3-yl)-1H-benzo[d]imidazole-4-carboxamide | | | |
| 52 | 2-(Bicyclo[2.2.1]heptan-2-yl)-7-hydroxy-N-((S)- | 0.43 | 2.3 | 19 |
| | piperidin-3-yl)-1H-benzo[d]imidazole-4-carboxamide | | | |
| 53 | 2-(Bicyclo[2.2.1]heptan-2-yl)-7-hydroxy-N- | 4.6 | 5.2 | 24 |
| | (adamantane-3-ylamino)-1H-benzo[d]imidazole-4- | | | |
| | carboxamide | | | |
| 54 | 2-(Thiophene-2-yl)-7-hydroxy-N-(adamantate-3- | 3.3 | 1.6 | 10 |
| | ylamino)-1H-benzo[d]imidazole-4-carboxamide | | | |
| 55 | N-(3-Aminocyclohexyl)-2-(bicyclo[2.2.1]heptan- | 2.9 | 9.1 | 74 |
| | 2-yl)-7-hydroxy-1H-benzo[d]imidazole-4- | | | |
| | carboxamide | | | |
| 56 | N-{[(cis)-4-Aminocyclohexyl]methyl}-2-(bicyclo | 10 | 30 | 61 |
| | [2.2.1]heptan-2-yl)-7-hydroxy-1H-benzo[d]imid- | | | |
| | azole-4-carboxatnide | | | |
| 57 | (S)-7-hydroxy-2-(5-(piperazin-1-yl)thiophen-2-yl)- | 1.1 | 1.1 | 33 |
| | N-(piperidin-3-yl)-1H-benzo[d]imidazole-4- | | | |
| | carboxamide | | | |
| 58 | (R)-7-hydroxy-N-(piperidin-3-ylmethyl)-2-(thio- | 13 | 24 | 21 |
| | phen-2-ylmethyl)-1H-benzo[d]imidazole-4- | | | |
| | carboxamide | | | |
| 59 | (S)-7-hydroxy-N-(piperidin-3-yl)-2-(thiophen-2- | 2.9 | 11 | 76 |
| | ylmethyl)-1H-benzo[d]imidazole-4-carboxamide | | | |
| 60 | (S)-7-hydroxy-N-(piperidin-3-ylmethyl)-2-(thio- | 49 | 42 | 33 |
| | phen-2-ylmethyl)-1H-benzo[d]imidazole-4- | | | |
| | carboxamide | | | |

“>100” in the table means over 100 microM.

### INDUSTRIAL APPLICABILITY

The present invention provides a novel 7-Hydroxy-benzoimidazole-4-yl-methanone derivative compound having PBK inhibitory effect. The compounds of the present invention may be used for pharmaceutical composition for inhibiting PBK. Such pharmaceutical compositions are suitable for treating or preventing cancer.

1. A compound represented by formula (I), or a salt, hydrate, solvate, or isomer thereof: 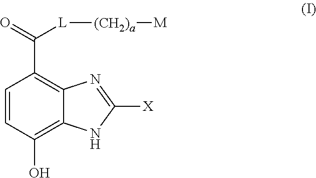
wherein
X is phenyl, thiophen-2-yl, furan-2-yl, cyclopropyl, cyclopentyl, phenyl-C₁-C₆ alkyl, thiophen-2-yl-C₁-C₆ alkyl, furan-2-yl-C₁-C₆ alkyl, cyclopropyl-C₁-C₆ alkyl, cyclopentyl-C₁-C₆ alkyl, or bicyclo[2.2.1]heptan-2-yl, wherein each group is optionally substituted by 1-3 substituent(s) that are each independently selected from a group A;
L is —NH—, or a single bond;
M is C₃-C₁₀ cycloalkyl, or a 3-8 membered saturated heterocyclic group, each optionally substituted by 1-3 substituent(s) that are each independently selected from the group A;
wherein the group A is selected from the group consisting of hydroxyl, oxo, nitro, cyano, amino, C₁-C₆ alkylamino, C₃-C₁₀ cycloalkylamino, amide, halogen, sulfamoyl, trifluoromethyl, p-toluenesulfonylamino, C₁-C₆ alkyl, C₃-C₁₀ cycloalkyl, C₁-C₆ alkoxy, C₁-C₆ alkoxycarbonyl, C₁-C₆ alkylcarbonylamino, C₁-C₆ alkylsulfonyl, C₁-C₆ alkylsulfonylamino, C₁-C₆ alkenyl, C₁-C₆ alkynyl, phosphoryl, carbonyl, carboxyl, and a 3-8 membered saturated heterocyclic group; and
a is an integer from 0 to 5.

2. The compound of claim 1, wherein M is piperidin-4-yl, piperidin-3-yl, piperidin-2-yl, piperazin-1-yl, pyrrolidin-3-yl, azetidin-3-yl, cyclohexyl, or adamantan-3-yl, which are each optionally substituted by 1 or 2 substituent(s) that are each independently selected from the group A.

3. The compound of claim 1, wherein X is thiophen-2-yl.

4. The compound of claim 1, wherein X is phenyl.

5. The compound of claim 1, wherein X is cyclopropyl.

6. The compound of claim 1, wherein X is cyclopentyl.

7. The compound of claim 1, wherein X is bicycle[2.2.1]heptan-2-yl.

8. The compound of claim 1, wherein X is 5-bromothiophen-2-yl.

9. The compound of claim 1, wherein X is 5-(piperazin-1-yl)thiophen-2-yl.

10. The compound of claim 1, wherein X is thiophen-2-ylmethyl.

11. The compound of claim 1, selected from the group consisting of:
2-Cyclopropyl-4-hydroxy-N-(piperidin-4-ylmethyl)-1H-benzo[d]imidazole-7-carboxamide,
2-Cyclopropyl-4-hydroxy-N-(piperidin-3-yl-methyl)-1H-benzo[d]imidazole-7-carboxamide,
2-Cyclopropyl-4-hydroxy-N-(piperidin-2-ylmethyl)-1H-benzo[d]imidazole-7-carboxamide,
2-Cyclopropyl-4-hydroxy-N-(1-methylpiperidin-3-yl)-1H-benzo[d]imidazole-7-carboxamide,
(S)-2-cyclopropyl-4-hydroxy-N-(piperidin-3-yl)-1H-benzo[d]imidazole-7-carboxamide,
2-Cyclopropyl-4-hydroxy-N-(piperidin-4-yl)-1H-benzo[d]imidazole-7-carboxamide,
2-Cyclopropyl-4-hydroxy-N-(piperidin-3-yl)-1H-benzo[d]imidazole-7-carboxamide,
2-Cyclopropyl-4-hydroxy-N-(pyrrolidin-3-yl)-1H-benzo[d]imidazole-7-carboxamide,
N-(azetidin-3-ylmethyl)-2-cyclopropyl-4-hydroxy-1H-benzo[d]imidazole-7-carboxamide,
2-Cyclopentyl-4-hydroxy-N-(piperidin-2-ylmethyl)-1H-benzo[d]imidazole-7-carboxamide,
2-Cyclopentyl-4-hydroxy-N-(piperidin-3-ylmethyl)-1H-benzo[d]imidazole-7-carboxamide,
(S)-2-Cyclopentyl-4-hydroxy-N-(piperidin-3-yl)-1H-benzo[d]imidazole-7-carboxamide,
(S)-4-Hydroxy-2-phenyl-N-(piperidin-3-yl)-1H-benzo[d]imidazole-7-carboxamide,
4-Hydroxy-2-phenyl-N-(piperidin-2-ylmethyl)-1H-benzo[d]imidazole-7-carboxamide,
4-Hydroxy-2-phenyl-N-(piperidin-3-ylmethyl)-1H-benzo[d]imidazole-7-carboxamide,
7-Hydroxy-N-(4-hydroxycyclohexyl)-2-(thiophen-2-yl)-1H-benzo[d]imidazole-4-carboxamide,
(7-hydroxy-2-[thiophen-2-yl]-1H-benzo[d]imidazol-4-yl)(piperazin-1-yl)methanone,
7-Hydroxy-N-(piperidin-3-yl)-2-(thiophen-2-yl)-1H-benzo[d]imidazole-4-carboxamide,
7-Hydroxy-N-[2-(piperazin-1-yl)ethyl]-2-(thiophen-2-yl)-1H-benzo[d]imidazole-4-carboxamide,
(R)-7-Hydroxy-N-(piperidin-3-yl)-2-(thiophen-2-yl)-1H-benzo[d]imidazole-4-carboxamide,
(S)-7-Hydroxy-N-(piperidin-3-yl)-2-(thiophen-2-yl)-1H-benzo[d]imidazole-4-carboxamide,
7-Hydroxy-N-(piperidin-3-ylmethyl)-2-(thiophen-2-yl)-1H-benzo[d]imidazole-4-carboxamide,
7-Hydroxy-N-(piperidin-4-yl)-2-(thiophen-2-yl)-1H-benzo[d]imidazole-4-carboxamide,
7-Hydroxy-N-(1-methylpiperidin-3-yl)-2-(thiophen-2-yl)-1H-benzo[d]imidazole-4-carboxamide,
7-Hydroxy-N-(piperidin-4-ylmethyl)-2-(thiophen-2-yl)-1H-benzo[d]imidazole-4-carboxamide,
N-(Azetidin-3-ylmethyl)-7-hydroxy-2-(thiophen-2-yl)-1H-benzo[d]imidazole-4-carboxamide,
7-Hydroxy-N-(pyrrolidin-3-yl)-2-(thiophen-2-yl)-1H-benzo[d]imidazole-4-carboxamide,
7-Hydroxy-N-(piperidin-2-ylmethyl)-2-(thiophen-2-yl)-1H-benzo[d]imidazole-4-carboxamide,
7-Hydroxy-N-(pyrrolidin-3-ylmethyl)-2-(thiophen-2-yl)-1H-benzo[d]imidazole-4-carboxamide,
N-(4-Aminocyclohexyl)-7-hydroxy-2-(thiophen-2-yl)-1H-benzo[d]imidazole-4-carboxamide,
2-(Bicyclo[2.2.1]heptan-2-yl)-7-hydroxy-N-(piperidin-3-ylmethyl)-1H-benzo[d]imidazole-4-carboxamide,
2-(Bicyclo[2.2.1]heptan-2-yl)-7-hydroxy-N-(piperidin-3-yl)-1H-benzo[d]imidazole-4-carboxamide,
(S)-tert-Butyl 3-(2-(5-bromothiophen-2-yl)-7-hydroxy-1H-benzo[d]imidazole-4-carboxamido)piperidine-1-carboxylate,
(S)-2-(5-bromothiophen-2-yl)-7-hydroxy-N-(piperidin-3-yl)-1H-benzo[d]imidazole-4-carboxamide,
2-(Bicyclo[2.2.1]heptan-2-yl)-7-hydroxy-N-((S)-piperidin-3-yl)-1H-benzo[d]imidazole-4-carboxamide,
2 (Bicyclo[2.2.1]heptan-2-yl)-7-hydroxy-N-(3-aminoadamantane-1-yl)-1H-benzo[d]imidazole-4-carboxamide,
2-(Thiophene-2-yl)-7-hydroxy-N-(3-aminoadamantane-1-yl)-1H-benzo[d]imidazole-4-carboxamide),
N-(3-Aminocyclohexyl)-2-(bicyclo[2.2.1]heptan-2-yl)-7-hydroxy-1H-benzo[d]imidazole-4-carboxamide,
N-{[(cis)-4-Aminocyclohexyl]methyl}-2-(bicyclo[2.2.1]heptan-2-yl)-7-hydroxy-1H-benzo[d]imidazole-4-carboxamide,
(S)-7-hydroxy-2-(5-(piperazin-1-yl)thiophen-2-yl)-N-(piperidin-3-yl)-1H-benzo[d]imidazole-4-carboxamide,
(R)-7-hydroxy-N-(piperidin-3-ylmethyl)-2-(thiophen-2-ylmethyl)-1H-benzo[d]imidazole-4-carboxamide,
(S)-7-hydroxy-N-(piperidin-3-yl)-2-(thiophen-2-ylmethyl)-1H-benzo[d]imidazole-4-carboxamide, and
(S)-7-hydroxy-N-(piperidin-3-ylmethyl)-2-(thiophen-2-ylmethyl)-1H-benzo[d]imidazole-4-carboxamide.

12. A method for preparing a compound of claim 1 which comprises the steps of:
contacting a carboxyalkyl substituted aniline derivative with a nitrile in the presence of an acid to form an intermediate amidine;
cyclizing the intermediate amidine to form a benzimidazole derivative having a carboxyalkyl;
saponifying the carboxyalkyl of the benzimidazole derivative to form a carboxylic acid; and
contacting the carboxylic acid of the benzimidazole derivative with an amine derivative, to obtain the compound of claim 1.

13. A pharmaceutical composition comprising at least one compound of claim 1 and a pharmaceutically acceptable carrier.

14. The pharmaceutical composition of claim 13 which is available for preventing or treating PBK dependent diseases.

15. The pharmaceutical composition of claim 14, wherein the PBK dependent disease is cancer.

16. A PBK inhibitor comprising at least one compound of claim 1.

17. A method for treating a PBK dependent disease in a subject, comprising administering to said subject an effective amount of a compound of claim 1.

18. (canceled)
